(19) United States
(12) Patent Application Publication
(10) Pub. No.: US 2013/0196880 A1
(43) Pub. Date: Aug. 1, 2013

(54) PATTERNED DEVICES AND METHODS FOR DETECTING ANALYTES

(71) Applicant: Ventana Medical Systems, Inc., Tucson, AZ (US)

(72) Inventors: Stacey Stanislaw, Tucson, AZ (US); Heather Lewis, Tucson, AZ (US); Lei Tang, Oro Valley, AZ (US); Lizhen Pang, Tucson, AZ (US); Hong Ni, Austin, TX (US)

(73) Assignee: VENTANA MEDICAL SYSTEMS, INC., Tucson, AZ (US)

(21) Appl. No.: 13/746,455

(22) Filed: Jan. 22, 2013

Related U.S. Application Data
(60) Provisional application No. 61/591,543, filed on Jan. 27, 2012.

(51) Int. Cl. C40B 40/06; C40B 40/12; C40B 40/10; C12Q 1/68; G01N 33/68

(52) U.S. Cl. CPC C40B 40/06; C12Q 1/6837; G01N 33/6803; C40B 40/10; C40B 40/12
USPC 506/16; 506/18

(57) ABSTRACT

Disclosed herein are devices for the detection of target molecules and methods for their use and production. The disclosed devices may include optically decipherable patterns that facilitate an understanding of binding events between a target molecule and a detection molecule. In one example, a device includes a macroscopic pattern constructed using microscopic elements that can be chromogenically developed to memorialize a binding event. In another example, a device includes characters that can be chromogenically developed using an automated slide staining instrument to memorialize a binding event.

### CROSS REFERENCE TO RELATED APPLICATION

This application claims the benefit of U.S. Provisional Application No. 61/591,543, filed Jan. 27, 2012, which is incorporated herein by reference in its entirety.

### FIELD

The present disclosure relates to devices having an array of detection molecules fabricated thereon and the use of those devices to detect target molecules.

### BACKGROUND

Nucleic acid arrays (also known as oligonucleotide arrays, DNA microarrays, DNA chips, gene chips, or biochips) have become powerful analytical tools. A nucleic acid array is essentially a systematic distribution of oligonucleotides on a surface, for example, in rows and columns. Oligonucleotides can be either physically or covalently adhered to a surface. One approach for physically adhering oligonucleotides to a surface involves drying oligonucleotide solutions as they contact the surface. After drying or otherwise fixing, the oligonucleotides are confined in a “spot” on the surface. The drying approach began with the production of very low density arrays called “dot blots.” Dot blots can be made by manually depositing drops of oligonucleotides on a solid surface and drying. Most dot blots involve fewer than about 20 different oligonucleotides spots arranged in rows and columns. Advancing past dot blots, microspotting approaches used mechanical or robotic systems to create a multiplicity of microscopic spots. The small size of the spots enabled much higher dot densities. For example, microspotting was used to deposit tens of thousands of spots onto a microscope slide. According to a different approach, oligonucleotides have been directly synthesized on a substrate or support. Maskless photolithography and digital optical chemistry techniques are techniques for directly synthesizing nucleic acids on a support; these approaches have been used to generate very high density arrays (for example, U.S. Pat. No. 7,785,863). Similarly, maskless photolithography has been used to manufacture peptide arrays (see, for example, Singh-Gasson et al. “Maskless fabrication of light-directed oligonucleotide microarrays using a digital micromirror array” Nat Biotechnol 1999, 17:974-978. Digital optical chemistry has been used that create arrays with millions of discrete areas each containing a population of unique oligonucleotides.

Nucleic acid and peptide arrays include an array of areas (referred to as “dots” herein) on a substrate surface, each area designated for a particular oligonucleotide or peptide. The “array density” is essentially the number of rows and columns of dots distributed in a given area. A high density array has a larger number of rows and columns in a given area. As the nucleic acid and peptide array industries have developed, the availability of high density arrays has also increased. As the number of dots in a given area increases, the size of each dot is reduced. For example, one dot in an array having millions of unique oligonucleotides or peptides distributed across the area of a microscope slide would be approximately 100 μm². The small size of this dot creates technical challenges in reading and understanding the results of using the array. For example, while a 100 μm² dot may be visually observed in isolation, humans cannot visually resolve two or more 100 μm² dots in close proximity without magnification. Thus, the manufacture and use of high density arrays has advanced to the stage that users can no longer read the array visually. Because the arrays include vast numbers (millions) of closely arrayed dots in a small area, sophisticated imaging devices detect signals from the array and software is used to interpret the data. Furthermore, the small size of the dot requires highly sensitive detection. Fluorescence imaging, being a highly sensitive technique, has become the standard approach for detecting hybridization events. Fluorescence imaging of these arrays generally uses microscopes equipped with filters and cameras. Fluorescence generally cannot be visually resolved without the aid of these devices. The highly complex fluorescence images are processed using software because the volume of data is high and its presentation is not cognizable. For example, U.S. Pat. No. 6,090,555 to Fiekowsky, et al. describes a complex process involving computer assisted alignment and deconvolution of fluorescence images acquired from a nucleic acid array. While the ability to perform massively parallel genomic or proteomic investigations is of great value, nucleic acid and peptide arrays have been limited in applicability by the difficulty in detecting and deciphering binding events. Furthermore, the use of fluorescence creates many hurdles to the general applicability of arrays due to fluorescence signals degrading over time and the complexity of the accompanying fluorescence detection hardware.

### SUMMARY

The present disclosure relates to a device and a method of using the device to detect target molecules, the device including an oligonucleotide or peptide array. The device includes a plurality of binding molecules bound to a substrate surface. The binding molecules are designed to bind to a target molecule. Binding of the target and the binding molecules can be identified through examination of the device. In some embodiments, the device enables the detection of a hybridization event between a target nucleic acid and an immobilized oligonucleotide. In other embodiments, the device enables the detection of a binding event between a target polypeptide and an immobilized peptide.

In illustrative embodiments, a device comprises a substrate with at least one substrate surface, and a plurality of immobilized oligonucleotides or peptides bound to the substrate surface, wherein the plurality of immobilized oligonucleotides or peptides are patterned on the substrate surface to form at least one optically decipherable pattern. In one embodiment, the at least one optically decipherable pattern is a glyph rendered from a character selected from the Universal Character Set, defined by the International Standard ISO/IEC 10646. For example, the glyphs may represent characters from Unicode Standard 6.0 (The Unicode Consortium. The Unicode Standard, Version 6.0.0, defined by: The Unicode Standard, Version 6.0 (Mountain View, Calif.: The Unicode Consortium, 2011. ISBN 978-1-936213-01-6; herein referred to as “Unicode”). Unicode and the Universal Character set ISO/IEC 10646 are substantially harmonized. In one embodiment, the plurality of immobilized oligonucleotides includes a first immobilized oligonucleotide that is sufficiently complimentary to a first target nucleotide sequence and a second immobilized oligonucleotide that is sufficiently complimentary to a first control nucleotide sequence. In another embodiment, the first immobilized oligonucleotide is patterned on the substrate surface to form a first character and the second immobilized oligonucleotide is patterned on the substrate surface to form a second character.

In one aspect, the optically decipherable pattern is of the type which enables recognition of binding events without reliance on sophisticated imaging devices. The immobilized oligonucleotides or peptides may be designed to be complimentary to one or more target or control nucleotide sequences or proteins. For example, a first immobilized oligonucleotide may be patterned on the substrate surface to form a first character and the second immobilized oligonucleotide is patterned on the substrate surface to form a second character, or glyph rendered therefrom. As such, hybridization of the first target may be made apparent by visualization of the first character and hybridization of the second target may be made apparent by visualization of the second character. Hybridization events associated with the control oligonucleotides may be made apparent by visualization of one or more other characters.

In illustrative embodiments, characters comprise a plurality of dots, the plurality of dots having at least one microscopic dimension. For example, the microscopic dimension may be between about 1 μm and about 500 μm. In one embodiment, a plurality of dots are patterned on the substrate surface at a linear density of between about 3 and about 4000 dots per inch. In another embodiment, at least about 75% of the plurality of immobilized oligonucleotides or peptides bound to the substrate surface within each of the plurality of dots have identical nucleic acid sequences or amino acid sequences. In yet another embodiment, the plurality of immobilized oligonucleotides bound to the substrate surface within each of the at least one character are sufficiently complimentary to a first target nucleotide sequence. In another embodiment, the plurality of immobilized oligonucleotides bound to the substrate surface within the at least one glyph are tiled across the first target nucleotide sequence. For example, the immobilized oligonucleotides are tiled across the first target nucleotide sequence in increments of 1 to 50 base pairs. In one embodiment, the first target nucleotide sequence is between about 10 and about 500 base pairs in sequence length and the immobilized oligonucleotides are between about 10 and 100 base pairs in length.

In illustrative embodiments, the plurality of immobilized oligonucleotides or peptides are patterned on the substrate surface to form at least one optically decipherable pattern. In one embodiment, the optically decipherable pattern is an information code. In another embodiment, the information code is an optical machine-readable representation of data. In yet another embodiment, the information code is a 1-dimensional data code or a 2-dimensional data code. In one embodiment, the pattern is compatible with automatic identification and data capture. For example, the pattern may be readable with optical character recognition, optical mark recognition, or bar code recognition.

In illustrative embodiments, a method of analyzing a sample using an array device comprises contacting the array device with a sample solution, contacting the array device with an antibody solution, the antibody solution comprising an antibody conjugated to an enzyme, contacting the array device with a chromogenic detection solution, the chromogenic detection solution comprising a compound that interacts with the enzyme to deposit a chromogenic substance on the array device to form at least one optically decipherable pattern, and deciphering the optical pattern. In one embodiment, target oligonucleotides hybridize with immobilized oligonucleotides. In another embodiment, target polypeptides interact with immobilized peptides. In another embodiment, the target oligonucleotides or polypeptides are hapten-labeled and contacting the array device with the sample solution includes labeling the immobilized oligonucleotides or peptides with the hapten. In another embodiment, the antibody solution includes an anti-hapten antibody and contacting the array device with the antibody solution includes binding the anti-hapten antibody to the hapten-labeled oligonucleotide. In yet another embodiment, deciphering the optical pattern includes recognizing the pattern as a character selected from the Universal Character Set, defined by the International Standard ISO/IEC 10646 and relating the at least one pattern to a meaning associated with the plurality of immobilized oligonucleotides or peptides. In one embodiment, deciphering the optical pattern includes using automated identification and data capture, wherein a computer is used to relate the at least one pattern to a meaning associated with the plurality of immobilized oligonucleotides. In another embodiment, contacting the array device with the chromogenic detection solution, contacting the array device with the antibody solution, and/or contacting the array device with a sample solution, is performed by an automated instrument. In another embodiment, the automated instrument is an automated slide staining instrument.

Contacting the array device with a sample solution enables the target molecules within the sample solution to bind with the binding molecule immobilized on the substrate. If the target compound is an oligonucleotide (e.g. haptenated single-stranded DNA), then contacting the array device with a sample solution enables the target oligonucleotide to hybridize to an immobilized oligonucleotide. If the target compound is a polypeptide (e.g. haptenated protein, antibody, or enzyme), then contacting the array device with a sample solution enables the target polypeptide to interact with immobilized peptide. Subsequent to binding between the target molecule and the immobilized molecule, a chromogenic detection approach may be applied to make the detection event apparent and to memorialize the detection event. For example, an anti-hapten antibody and enzyme conjugate can be allowed to bind to the hapten followed by enzymatic chromogenic deposition.

In illustrative embodiments, a device for memorializing a hybridization event comprises a substrate with at least one substrate surface, a plurality of immobilized oligonucleotides or peptides bound to the substrate surface, a plurality of target oligonucleotides hybridized or polypeptides interacting with the plurality of immobilized oligonucleotides or peptides, the plurality of target oligonucleotides or polypeptides including labels, and a plurality of stable chromogenic depositions localized proximally to the plurality of immobilized oligonucleotides or peptides, wherein the labels direct the plurality of stable depositions to form an optically decipherable pattern on the substrate, the optically decipherable pattern memorializing the detection event. In one embodiment, the labels include a hapten. In another embodiment, the labels include a hapten bound to an enzyme-conjugated anti-hapten antibody. In another embodiment, the enzyme-conjugated anti-hapten antibody includes a horseradish peroxidase (HRP) or alkaline phosphatase (AP) enzyme. In one embodiment, the plurality of stable chromogenic depositions are immunohistochemical stains. In yet another embodiment, the stable chromogenic depositions are selected from silver, nickel, DAB (diaminobenzidine), fast red (4-chloro-2-methylbenzenediazonium/3-hydroxy-2-naphthoic acid 2,4-dimethylanilide phosphate), fast blue (o-dianisidine bis(diazotized) zinc double salt), and BCIP/NBT (5-Bromo-4-chloro-3-indolyl phosphate/nitro blue tetrazolium chloride). In one embodiment, the plurality of target oligonucleotides includes a cocktail of at least two uniquely specific DNA probes. In another embodiment, the optically decipherable pattern comprises a plurality of microscopic array features having at least one dimension less than about 200 μm. In another embodiment, the optically decipherable pattern includes at least one feature having a length of between about 200 μm and 75 millimeters. The device enables memorialization of the hybridization event because the stable depositions on the device provide an archival record that a specific binding event occurred. The archival record is stable for multiple examinations and storage and is additionally not reliant on sophisticated imaging analysis and software which may or may not be available after significant storage periods.

### BRIEF DESCRIPTION OF THE DRAWINGS

The detailed description refers to the accompanying figures in which:

FIG. 1(A-B) are partial perspective views of embodiments of the present invention showing (A) immobilized oligonucleotides or (B) immobilized peptides bound to a substrate surface, the views portraying alternative highly magnified views of FIG. 2;

FIG. 2. is a magnified top plan view of the device of FIG. 3 showing a microscopic pattern comprising a multiplicity of dots arranged in an array;

FIG. 3. is a top plan view of a device according to an illustrative embodiment of the present invention showing a plurality of optically decipherable patterns;

FIG. 4 is a map assigning values for the positional pattering of the device of FIG. 3;

FIG. 5 is a top plan view of a device according to an alternative embodiment of the present invention showing a plurality of optically decipherable patterns

FIG. 6 is a schematic showing the alternative placements of a targeting segment within an immobilized oligonucleotide;

FIG. 7 is a magnified top plan view of a device showing a microscopic pattern comprising a multiplicity of dots arranged in an array and further including a schematic showing how nucleic acid sequences may be tiled across patterns;

FIG. 8 is a schematic of a method of using a device on an analyzer to generate a decipherable pattern;

FIG. 9(A-D) show diagramatically a progression of a method according to one embodiment of the present invention in which (A) shows an immobilized oligonucleotide on a substrate surface, (B) shows a complementary target oligonucleotide hybridized to the immobilized oligonucleotide, the oligonucleotide being haptenated with a first hapten, (C) shows an anti-hapten antibody conjugate with an enzyme, and (D) shows an enzymatically deposited chromogenic substance deposited on the substrate surface;

FIG. 10(A-E) show diagramatically a progression of a method according to one embodiment of the present invention in which (A) shows an immobilized oligonucleotide on a substrate surface, (B) shows a complementary oligonucleotide hybridized to the immobilized oligonucleotide, the oligonucleotide being haptenated with a first hapten, (C) shows an anti-hapten antibody conjugate with a secondary hapten bound to the first hapten, (D) shows an anti-hapten antibody-enzyme conjugate bound to the second hapten, and (E) shows an enzymatically deposited chromogenic substance deposited on the substrate surface;

FIG. 11 is a top plan view of a device according to an embodiment of the present invention showing machine readable optically decipherable patterns;

FIG. 12(A-B) are photograph of a device according to one embodiment showing a magnified view of a first portion (A) and a magnified second portion (B), the device having a number of characters in an optically decipherable pattern;

FIG. 13 is a highly magnified photograph of a device showing the microscopic array elements, dots, chromogenically stained black and red;

FIG. 14 is a magnified photograph of a portion of a device showing the differential staining apparent for different concentrations of target molecules; and

FIG. 15 is a magnified photograph of a gray scale bar that includes varying ratios of positive and negative control features.

### DETAILED DESCRIPTION

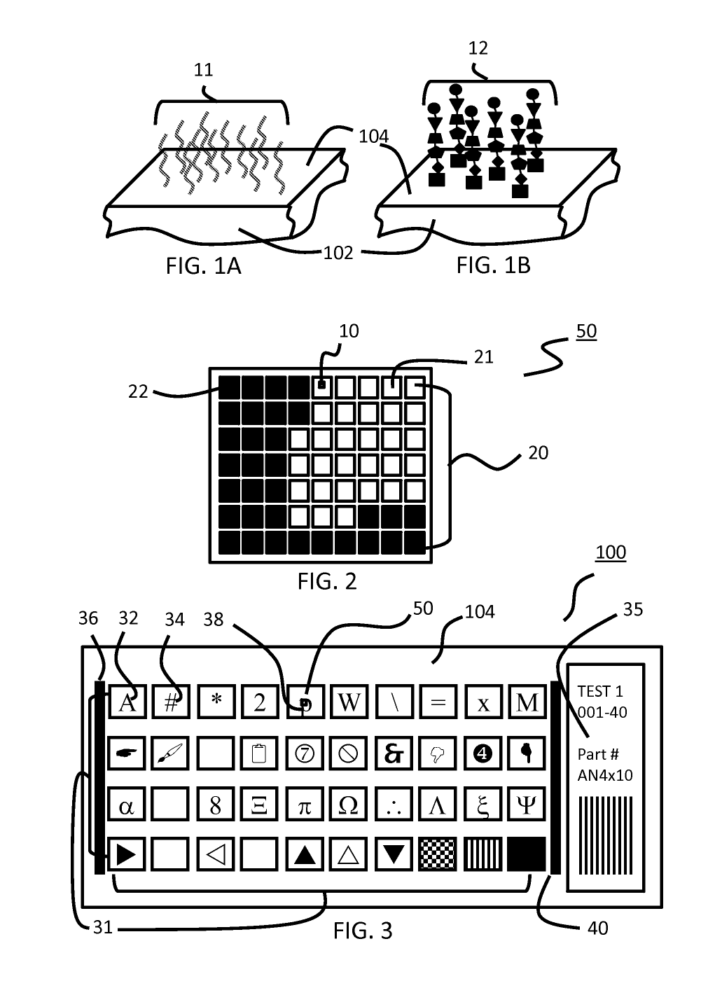

Referring now to FIGS. 1-3, FIG. 3. is a top plan view of a device 100 according to an illustrative embodiment of the present disclosure showing a plurality of optically decipherable patterns 31. FIG. 2. is a highly magnified top plan view (approximately 40×) of a portion 50 of the device of FIG. 3. FIG. 1(A-B) are alternative highly magnified partial perspective views of a portion 10 of FIG. 2. FIGS. 1-3 show device 100 comprising a substrate 102 with at least one substrate surface 104. A plurality of immobilized oligonucleotides 11 or peptides 12 are bound to substrate surface 104. Immobilized oligonucleotides 11 or peptides 12 are patterned on substrate surface 104 according to patterns on a microscopic and macroscopic scale. FIG. 2 shows a portion 50 of a microscopic pattern comprising a multiplicity of dots arranged in an array 20. For example, FIG. 2 shows portion 50 having a pattern of 7 rows and 9 columns of dots. The array extends for substantial lengths in both dimensions, portion 50 is shown merely as an example. Array 20 is depicted as having two distinct immobilized oligonucleotide types, one is depicted as a hollow square 21 and the other is depicted as a solid square 22. The solid and hollow squares indicate that some property (e.g. sequence, length) of the immobilized oligonucleotides or peptides in those areas are distinct. Referring now to FIG. 3, optically decipherable patterns 31 are shown on substrate surface 104. The optically decipherable patterns are macroscopic and comprise a plurality of characters. Illustratively, the device also includes a label 35. For example, label 35 may be a printed product identification label with a product description, designation, and a bar code. Illustratively, the printed label is for identification purposes and may or may not come into contact with any solutions or reagents described herein.

The macroscopic patterns of FIG. 3 are formed using the underlying microscopic pattern (e.g. multiplicity of dots arranged in array 20 as shown in FIG. 2). In particular, device 100 includes the optically decipherable patterns of a character A 32 (Unicode 0041 Latin Capital Letter A), a #34 (Unicode 0023 Number Sign), or a heavy vertical 36 and 40 (Unicode 2503 Box Drawing Heavy Vertical). A lowercase letter p 38 (Unicode 0070 Latin Small Letter P) is an optically decipherable pattern that includes a portion 50, a magnified view thereof is shown in FIG. 2. The pattern of character p 38 is created on surface 104 using the dots shown in array 20. According to this example, array 20 is an underlying pattern upon which optically decipherable patterns 31 are formed. Character p 38, as shown in FIG. 3, is made of dots, as shown in FIG. 2, the dots comprising immobilized oligonucleotides 11 or peptides 12, as shown in FIG. 1(A-B). As an example of how the oligonucleotides are patterned, solid square 22 includes oligonucleotides or peptides having a different character than the oligonucleotides or peptides immobilized in hollow square 21. The difference between the oligonucleotides or peptides enables the dots to be distinguished. In one embodiment, the difference can be that one oligonucleotide or peptide participates in a detectable binding event while the other does not (i.e. the immobilized oligonucleotides or peptides in each have distinct sequences). In another embodiment, the difference can be that diverse detection chemistries are elicited by distinct binding events. Accordingly, the difference between the oligonucleotides or peptides can be distinguished by a difference in binding state (i.e. binding vs. non-binding) or a difference in binding character (e.g. binding of a first type results in a blue colored pattern and binding of a second type results in a red colored pattern).

One purpose of a device according to the present invention is the detection of one or more target compounds. One type of target compound of particular interest is target nucleic acids or target oligonucleotides. Another type of target compound of particular interest is target polypeptides. For embodiments of the present invention including immobilized oligonucleotides, target nucleic acids would commonly be understood to be the target molecule type. However, those of ordinary skill in the art appreciate that immobilized oligonucleotides provide a binding partner for oligonucleotide-binding moiety conjugates that are capable of detecting a variety of other target compounds. For example, using the immobilized oligonucleotide, an antibody-oligonucleotide conjugate could be immobilized on the device to transform the device into an antibody microarray. An antibody microarray could be used to detect a protein target of interest. Similarly, embodiments that include immobilized peptides, the target molecule type could include antibodies, proteins, or enzymes. However, the underlying peptides could also be modified by using conjugates of the peptide binding moiety and a molecular targeting moiety. Furthermore, while the present disclosure specifically discloses immobilized oligonucleotides and peptides, those are merely exemplary immobilized detection moieties. There are many other useful immobilized detection moieties that may be incorporated into a device as described herein, without departing from the concept as disclosed herein. For example, the detection moieties may include aptamers, ligands, chelators, carbohydrates, and to man-made equivalents thereof.

In illustrative embodiments, carbohydrates could be used as a ligand or in other ways to bind other carbohydrates or whole bacterial cells. For example, a device including an array of carbohydrates could be used to detect specific bacteria. In this embodiment, the chromogenic detection solution could be used to deposit chromogen according to the localization of those bacteria. For example, selecting strain specific binding carbohydrates would enable the identification of strain by reading the pattern on the device. As discussed herein, with respect to oligonucleotides and peptides, carbohydrates could be patterned on the surface using digital optical chemistry or by using oligonucleotide-carbohydrate conjugates which could couple to a device comprising an array of oligonucleotides.

In illustrative embodiments, the test composition (i.e. sample to be analyzed) can be any suitable composition having an analyte of interest (e.g. molecular, viral, bacterial). In one embodiment, the test composition can be any suitable composition for detecting a target oligonucleotide (e.g. a cocktail of labeled oligonucleotides; a cocktail of nucleic acid probes; an unknown biological or environmental sample; a resultant solution from a previous laboratory process or test such as an eluent or an aliquot from a chromatography process, an amplified nucleic acid solution such as a product of a polymerase chain reaction (PCR), a digest of treated cells, a digest of untreated cells, a digest of treated tissue, a digest of untreated tissue, or DNA from plasmid amplification). Treatment of the cells or tissues can be chemical (e.g., osmotic treatment, membrane disrupting chemicals), mechanical (e.g., sonication, homogenization), or combinations thereof. In certain embodiments, the test composition can be a biological specimen containing DNA (for example, genomic DNA), RNA (including mRNA), protein, or combinations thereof, obtained from a subject. Examples of biological specimens include, but are not limited to, chromosomal preparations, peripheral blood, urine, saliva, tissue biopsy, surgical specimen, bone marrow amniocentesis samples, cytological samples, and autopsy material. In some examples, the biological specimen can comprise genomic DNA, a cytogenetic preparation, a manipulated sample (e.g., by fixing using a fixing reagent such as formalin), or combinations thereof. In some embodiments, the test composition comprises one or more labeled nucleic acids (e.g., RNA or single stranded DNA). In certain embodiments, the test composition comprises one or more hapten-labeled oligonucleotide probes designed for use in in situ hybridization assays, in chromogenic in situ hybridization assays, in fluorescent in situ hybridization assays, or in silver in situ hybridization assays. In other embodiments, one or more hapten-labeled oligonucleotide probes detect molecular marker(s) of certain cancers.

A nucleic acid is a deoxyribonucleotide, ribonucleotide, or synthetic equivalent polymer in either single or double stranded form, and unless otherwise limited, encompassing analogs of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A nucleotide is one monomer in a polynucleotide or in an oligonucleotide. A nucleotide sequence refers to the sequence of bases in a polynucleotide or in an oligonucleotide. The term “oligo” is sometimes used to refer to an oligonucleotide. The term “X-mer” refers to a single stranded nucleic acid with X number of bases; for example, 10-mer is a single stranded nucleic acid with 10 bases. A nucleic acid “segment” is a nucleic acid that has only a subsequence of a larger nucleic acid.

The term nucleotide also includes synthetic analogs that mimic the interactions of natural nucleotides. For example, the nucleotide may be a morpholino or locked nucleic acid. As such, a device with an array of mimic molecules may be capable of detecting the presence of synthetic nucleic acid mimics. In another embodiment, a device with an array of mimic molecules may be used to detect conjugates of mimics coupled to other biomolecules or may be transformed from a mimic molecule array to a biomolecular array through the conjugates. like we mention previously, to make those conjugates addressable to a DNA array.

A target oligonucleotide can be any desired oligonucleotide, such as but not limited to single stranded nucleic acids, double stranded nucleic acids, single stranded DNA, or single stranded RNA (e.g., messenger RNA, transfer RNA or ribosomal RNA). In some embodiments, the target oligonucleotide can be a single stranded cDNA, a double stranded cDNA, or DNA from the genome of an organism (e.g., mitochondrial DNA or DNA from a chromosome). Other embodiments include DNA sequences (e.g., from the human genome) whose presence may be indicative of a disease or condition. Such a disease or condition includes but is not limited to cancer. In some embodiments, the target nucleic acid is a labeled nucleic acid or otherwise modified to facilitate detection when bound to the immobilized oligonucleotide. Labels and modifications can include, but are not limited to, fluorescent moieties, fluorogenic moieties, chromogenic moieties, haptens, affinity tags, luminescent reagents, electron capture reagents, light absorbing dyes, and radioactive isotopes. The label or modification can be directly detectable (e.g., optically detectable) or indirectly detectable (for example, via interaction with one or more additional molecules that are in turn detectable). In one embodiment, the label is a hapten. The length of the target oligonucleotide can be any suitable length, including but not limited to lengths of at least about 20 bases, or at least about 30 bases.

Binding is the association between two substances or molecules, such as the hybridization of one nucleic acid molecule (e.g., an immobilized oligonucleotide) to another (e.g., a target oligonucleotide). A nucleic acid molecule binds (sometimes referred to as specific binding) to a another nucleic acid molecule if a sufficient number of the molecule's nucleic acid base pairs are hybridized to another nucleic acid molecule to permit detection of that binding. Binding can be detected by any suitable procedure, such as but not limited to physical or functional properties of the binding complex. Physical methods of detecting the binding of sufficiently complementary strands of nucleic acid molecules include, but are not limited to, DNase I or chemical footprinting, gel shift and affinity cleavage assays, Northern blotting, dot blotting and light absorption detection procedures. In another example, the method involves detecting a signal, such as a detectable label, present on one or both nucleic acid molecules. Nonspecific binding can occur when some detection is present, but hybridization or base pairing between the two nucleic acids does not occur.

As used herein, a nucleic acid molecule is said to be “complementary” with another nucleic acid molecule (or is a “complement” of a nucleic acid sequence) if the two molecules share a sufficient number of complementary nucleotides to form a stable duplex or triplex when the strands bind (hybridize) to each other, for example by forming Watson-Crick, Hoogsteen, or reverse Hoogsteen base pairs. Stable binding occurs when a nucleic acid molecule (e.g., a uniquely specific nucleic acid molecule, a centromere nucleic acid molecule, a medically relevant nucleic acid molecule) remains detectably bound to a target nucleic acid (e.g., genomic target nucleic acid) under the required conditions. Complementarity is the degree to which bases in one nucleic acid molecule (e.g., a probe nucleic acid molecule) base pair with the bases in a second nucleic acid molecule (e.g., genomic target nucleic acid molecule). Complementarity is conveniently described by percentage, that is, the proportion of nucleotides that form base pairs between two molecules or within a specific region or domain of two molecules. For example, if 10 nucleotides of a 15 contiguous nucleotide region of a probe nucleic acid molecule form base pairs with a target nucleic acid molecule, that region of the probe nucleic acid molecule is said to have 66.67% complementarity to the target nucleic acid molecule.

“Sufficient complementarity” means that a sufficient number of base pairs exist between one nucleic acid molecule (or segment thereof) and a target nucleic acid sequence (or segment thereof) to achieve detectable binding. One treatment of the considerations involved in establishing binding conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Hybridization occurs when base pairs form between complementary regions of two strands of DNA, RNA, or between DNA and RNA, thereby forming a duplex molecule. The term hybridization may also be used to describe the interaction between nucleic acid mimics or between a mimic and a natural nucleic acid. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. The presence of a chemical which decreases hybridization (such as formamide) in the hybridization buffer will also determine the stringency (Sadhu et al., J. Biosci. 6:817-821, 1984). Calculations regarding hybridization conditions for attaining particular degrees of stringency are discussed in Sambrook et al., (1989) (Chap. 9 and 11). Hybridization conditions for ISH are also discussed in Landegent et al., Hum. Genet. 77:366-370, 1987; Lichter et al., Hum. Genet. 80:224-234, 1988; and Pinkel et al., Proc. Natl. Acad. Sci. USA 85:9138-9142, 1988. Those of ordinary skill in the art would be able to select the appropriate hybridization conditions for particular degrees of stringency.

The sequence identity (or similarity) between two or more nucleic acid sequences is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. The following are exemplary hybridization conditions for determining identity between oligonucleotides:

Very High Stringency (Detects Sequences that Share at Least 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 60% Identity)

Hybridization: 6×SSC at RT to 55° C. for 16-20 hours

Wash at least twice: 2×-3×SSC at RT to 55° C. for 20-30 minutes each.

Methods of alignment of sequences for comparison are well known in the art. Various programs and alignment algorithms are described in: Smith & Waterman, Adv. Appl. Math. 2:482, 1981; Needleman & Wunsch, J. Mot Biol. 48:443, 1970; Pearson & Lipman, Proc. Natl. Acad. Sci. USA 85:2444, 1988; Higgins & Sharp, Gene, 73:237-44, 1988; Higgins & Sharp, CAB/OS 5:151-3, 1989; Corpet et al., Nuc. Acids Res. 16:10881-90, 1988; Huang et al., Computer Appls. in the Biosciences 8, 155-65, 1992; and Pearson et al., Meth. Mot Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN may be used to compare nucleic acid sequences, while BLASTP may be used to compare amino acid sequences. If the two compared sequences share homology, then the designated output file will present those regions of homology as aligned sequences. If the two compared sequences do not share homology, then the designated output file will not present aligned sequences. The BLAST-like alignment tool (BLAT) may also be used to compare nucleic acid sequences (Kent, Genome Res. 12:656-664, 2002). BLAT is available from several sources, including Kent Informatics (Santa Cruz, Calif.) and on the Internet (genome.ucsc.edu).

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166/1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 15 consecutive nucleotides from an identified sequence as follows contains a region that shares 75.0 percent sequence identity to that identified sequence (that is, 15/20*100=75.0).

The specific interaction between a peptide and a protein is often referred to as “binding” but may also be referred to as “molecular recognition.” The magnitude of the interaction is often referred to as “affinity.” A peptide binds to a polypeptide if a sufficient portion of the polypeptide interacts with the peptide so that the peptide-polypeptide complex can be detected. A binding complex can be detected by any suitable procedure, such as detecting the physical or functional properties of the binding complex. Physical methods of detecting the complex includes, but is not limited to, target polypeptide specific antibody mediated detection of presence of target polypeptide protein co-localized to immobilized peptide. In another example, the method involves detecting a signal, such as a detectable label, present on either the peptide or the polypeptide. Nonspecific binding can occur when some detection is present, but no specific binding has occurred between the peptide and the polypeptide. Illustratively, nonspecific binding lacks sufficient affinity and specificity to be classified as molecular recognition. The presence of sufficient ionic, hydrogen, or van der Waal forces interactions are typically responsible for affording a complex its affinity. Stable binding occurs when an immobilized peptide binding molecule (e.g., peptide sequence used as antigen to generate a monoclonal antibody) remains detectably bound to a target peptide or polypeptide (e.g., monoclonal antibody designed to recognize a particular peptide sequence) under the conditions used by device conditions. Methods and examples of protein-protein interactions can be found in review by Phizicky and Fields, “Protein-Protein Interactions Methods for Detection and Analysis”, Microbiological Reviews, March 1995, p. 94-123 Vol. 59, No. 1.

Peptide-peptide or peptide-polypeptide binding occurs when sufficient interactions between the amino acids of each molecule interact sufficiently to form a binding complex. Intermolecular interactions can occur between natural amino acids, between non-natural amino acids, or mixtures of the same. Ligand-binding interactions and interactions between non-peptides and peptides can also result in the formation of a binding complex (e.g. myoglobin binding heme). Solution conditions such as pH, salt concentration, temperature, additives (e.g. surfactants), and analyte concentration may all effect the strength and stability of the binding between molecules. The state of the target molecule may also affect binding. For example, denatured target proteins may bind with differing affinity than native proteins to the same immobilized binding molecule. Similarly, digestion products of target proteins may also have distinct binding affinities than intact proteins. Approaches to testing the interactions between peptides and polypeptides have been described (Berggard et. al, “Review: Methods for the detection and analysis of protein-protein interactions” Proteomics 2007, 7, 2833-2842; Petsalaki et al. (2009) Accurate Prediction of Peptide Binding Sites on Protein Surfaces. Comput Biol 5(3): e1000335. Hertz et al., “PepDist: A New Framework for Protein-Peptide Binding Prediction based on Learning Peptide Distance Functions”, BMC Bioinformatics 2006, 7(Supplement 1)). Approaches to selecting test conditions for evaluating binding between particular immobilized binding molecules and target compounds are disclosed therein.

Both an immobilized peptide binding molecule and target molecule can include any suitable amino acid monomer. Amino acid monomers could include both naturally occurring proteinogenic amino acids (e.g. Lysine and Glycine), non-proteinogenic amino acids (e.g. carnitine and beta-Alanine), or even non-natural ones (e.g. O-methyl-tyrosine as reported by Wang et al. “Expanding the genetic code of Escherichia coli”. Science 2001, 292:498-500). The immobilized peptide can be any length suitable for the detection of the target molecule, including but not limited to lengths of at least about 6 monomers, at least about 8 monomers, at least about 10 monomers, or between about 6 and about 50 monomers. The minimum length suitable for detection may rely on the nature of the target molecule.

In illustrative embodiments, a device includes a plurality of immobilized binding molecules tiled across a target or control molecule (e.g. an epitope) within one pattern on the substrate surface. Tiling immobilized peptide sequences may be used to map protein epitopes to characterize antibody binding sites. For example, the tiling of binding molecule peptides across an antigenic sequence may advantageously provide a presentation of multiple similar, but distinct, amino acid sequences to a particular target molecule. Using tiling, it is possible to test which of sequences exhibit binding. As described herein, within each dot the immobilized peptide binding molecules are substantially identical. Furthermore, within a pattern, glyph, or character, the immobilized peptides may be organized such that each dot is specific to the same target molecule.

In illustrative embodiments, a device according to the present disclosure includes a microscopic pattern and a macroscopic pattern that is optically decipherable. In one embodiment, the optically decipherable pattern is a glyph rendered from a character selected from the Universal Character Set, defined by the International Standard ISO/IEC 10646. While various patterns can be used, characters from the Universal Character Set may be useful within the present invention because they either intrinsically carry meaning or can easily be assigned meaning by definition. In particular, since characters possess semantic value and have well-defined representations (e.g. through typefaces), they aid readability and enhance use of devices according to the present disclosure. A random pattern or pattern that is not optically decipherable requires that the multiplicity of separate signals making the pattern be mapped and analyzed. The use of characters from the Universal Character Set enables a determination of the binding events without reliance on data mapping.

For example, a reader considering device 100 of FIG. 3 could read the symbols shown in each row and associate meaning or a definition to each symbol. At a macroscopic scale, the information shown on device 100 could be categorized according to at least two levels of organization. In illustrative embodiments, the device includes two levels of macroscopic information organization. One manner of macroscopic information organization is positional patterning and the other is shape patterning. Device 100 includes both positional patterning and shape patterning. The positional patterning shown in FIG. 3 is a four-by-ten (4×10) array 31 of squares spaced between two heavy verticals 36, 40.

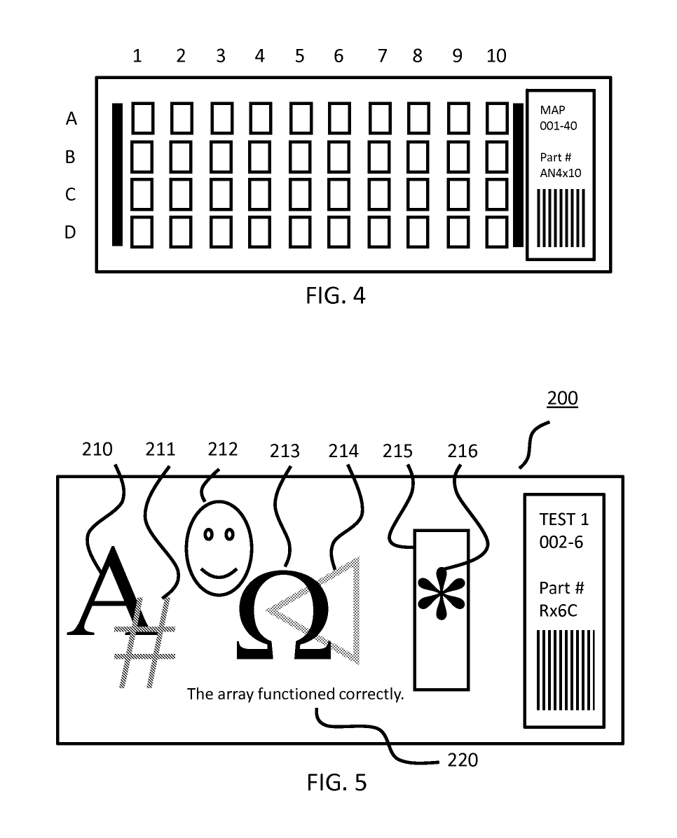

Referring now to FIG. 4, shown is a map providing understanding to the positional patterning for device 100. In an exemplary use of positional patterning, the binding of a first oligonucleotide target (EXA-1) could be developed in a manner described herein to be confirmed by the existence of a mark in position A-1. Examining device 100 in relation to the map shown in FIG. 4, it is evident there is a mark in position A-1. Using positional patterning, the binding of EXA-1 can be confirmed. In another example, the binding of a second oligonucleotide target (EXA-2) could be developed in a manner described herein to exhibit a mark in position B-3. Examining device 100 in relation to the map shown in FIG. 4, no marks are present at position B-3. Accordingly, the absence of EXA-2 can be determined by the absence of a mark at position B-3. Positional patterning does not rely on mark shape for identification of a binding event; instead, it relies on position and cognition of position. Device 100 also includes shape patterning and the enhanced readability that shaped patterning provides can be demonstrated by reference thereto. For example, the binding of EXA-1 can be determined by the presence of character A on the device. Without reference to any map, it is possible to confirm that EXA-1 is bound. Similarly, if the binding of EXA-2 was to be determined by the presence of a character D, the test composition would be determined as lacking EXA-2.

Positional patterning is well-known in the art. An array, by definition, is a pattern including a number of rows and columns. These rows and columns could be designated with letters or numbers so that each location is effectively mapped. However, as the density of arrays has increased, effectively identifying individual array elements (dots) has been increasingly difficult. For example, the use of a map is confounded because alignment between the map and the device is not always possible. To address these limitations, researchers have developed alignment tools which reduce error rates in translating microscopic elements to their map location (for example, U.S. Publication No. 2011/0268347). As this reference describes, the microarray field has used randomization of signal spots and randomized alignment spots to overcome alignment errors. The present disclosure describes the opposite approach using deliberate patterning and macroscopic patterning in unexpected ways to produce an array having superior usefulness within the scope of its intended use. In illustrative embodiments, a device according to the present disclosure includes layered patterning (i.e. microscopic patterning providing a basis for macroscopic patterning). In one embodiment, the device includes dots organized according to a pattern of characters. In another embodiment, the microscopic array underlies and defines the macroscopic patterns. In another embodiment, the macroscopic pattern conveys machine-readable optical patterns.

In addition to positional patterning, illustrative embodiments of the present invention include shape patterning. In one embodiment, shape patterning is a macroscopic patterning. As used herein, macroscopic refers to sizes that can be visually seen and/or read using the un-aided eye (e.g. under no magnification). Referring again to FIG. 3, examples of shape patterning are shown. In illustrative embodiments, shape patterning includes using characters. The term character is used to mean the smallest component of written language that has semantic value. It traditionally refers to the abstract meaning and/or shape, rather than a specific shape, though in code tables some form of visual representation is essential for the reader's understanding. The term “glyph,” or “glyph image,” is used to describe one or more images for displaying characters. One or more glyphs may be selected to depict a particular character (e.g. the character “i” is made up of two glyphs, one being the stem and the other being the dot). Glyphs are selected by a rendering engine during composition and layout processing to represent a character. As used herein, the term character is used interchangeably with glyph and glyph image to refer to a specific glyph image representing a component of written language that has semantic value.

Referring again to FIG. 3, heavy verticals 36 and 40 are glyphs rendered from characters and thus constitute shape patterning. In particular, the bars are glyphs representing characters from Unicode Standard 6.0 (Character 2503 Box Drawing Heavy Vertical). Whether referred to as glyphs, glyph images, or characters, the shape patterning shown in FIG. 3 is derived from Unicode characters. Unicode and the Universal Character set ISO/IEC 10646 are substantially harmonized. Referring now to FIG. 5, shown is a device 200 that does not include particular positional patterning rather includes primarily shape patterning. In particular, array 200 includes the following Unicode characters: A (210), # (211), [symbol] (212), Ω (213), [symbol] (214), [symbol] (215), and * (216). Additionally, shown is a sentence 220 that represents a possible pattern having both shape and positional patterning elements. According to this example, the position of the shapes on the array is not of importance and a map of the array is not necessary for deciphering the results. Instead, a lack of positional clarity does not diminish the readability of the pattern. FIG. 5 shows several characters with overlapping positions, yet it is still possible to decipher the presence of all characters. In illustrative embodiments, a device according to the present disclosure can be deciphered without use of a map. In one embodiment, the device includes overlapping symbols, for instance, to increase symbol density. In yet other embodiments, the use of characters permits extensive overlap because the shapes are recognizable even where a portion of the shape may be obscured by another character. While embodiments of the present invention use characters, partial characters, to the extent that they are cognizable as the character are within the scope of the present disclosure.

One advantage of shape patterning is that it may not require a map for deciphering. Microarray devices of the prior art require complex maps for data analysis in addition to the sophisticated instrumentation to read. As a particular advantage over those devices, embodiments of the present invention may be read without a map and without any instrumentation. The inclusion of shape patterning within the present invention provides advantages because the shapes enhance the readability of the device and enables the display of complex information in a simple decipherable format. The use of only position patterning essentially reduces the information gathered from a device to a binary language. For example, on a traditional microarray device, a binding event is either recognized or not by the presence of a signal at a particular location. While the binding event may also include a classification and a location (e.g. a red signal at location B-3), the signal does not carry significant semantic value. Using positional and shape patterning, the information content and information density can increase while making the device more readable. For example, it is easier for a literate person to read the sentence “The array functioned correctly.” than it would be for that person to read a series of zeros and ones (e.g. 0100110011000) that has been given the meaning “the array functioned correctly.” In illustrative embodiments, a device includes a first character and a second character. In one embodiment, the characters are different. In another embodiment, the first character and the second character are located in different areas on the substrate surface. In another embodiment, the device includes at least three characters, at least 10 characters, or at least 100 characters. In another embodiment, the device includes between about 3 and 10,000 characters. In yet another embodiment, the device includes between 5 and 100 characters. In yet another embodiment, the device includes between 20 and 1000 characters.

The number of symbols on the device relates to the information density that the device is built to include. The information density of the exemplary device shown in FIG. 5 is relatively low. The eight (8) symbols (the sentence being referred to as a single symbol) distributed across the array would be significantly lower than the current state of the art for commercially available arrays. Commercially available arrays are now including in excess of 4 million features (e.g. NimbleGen® Human CGH 3x1.4M Whole-Genome Tiling Arrays). However, device 200 can be deciphered without reliance on automated detection systems or the accompanying software for interpreting the detected signals. Instead, a user could determine whether a particular binding event occurred by simply referring to an instruction manual accompanying the device. For example, the instruction manual could include statements such as “the presence of the sentence, ‘The array functioned correctly.’ on the array indicates the detection chemistry performed properly” (i.e. the sentence may serve as a positive control). The instruction manual could further include sample-specific results. For example, the instruction manual could include a statement such as, “the presence of a ‘smiley face’ indicates the binding of nucleic acid sequence EXA-3.” The instructions could further provide “the presence of nucleic acid sequence EXA-4 may be indicated by the presence of a ‘frowning face.’” Accordingly, deciphering the device would include an appreciation that nucleic acid EXA-3 bound to array 200 while EXA-4 did not bind. While device 200 is shown with very low information density, the information density could be significantly enhanced by reducing the size of the shape patterns, incorporating a greater number of shape patterns across the device, or increasing the size of the array device.

In illustrative embodiments, immobilized binding moieties are organized on the substrate surface as a multiplicity of dots. As the term is used in the printing industry, a dot is the smallest area of a surface that a particular device can print. Because it is the smallest printable area, the composition of the ink at that location is typically homogeneous. In a similar manner, the dot of a microarray is typically the smallest area that can be controllably manufactured by the array manufacturing equipment. For example, state of the art arrays manufactured using digital optical chemistry techniques have been used to generate very high density arrays of oligonucleotides arranged as a multiplicity of dots. Each of the dots resulting from this manufacturing process can currently be as small as about 1 to about 100 μm² without unduly sacrificing accuracy or reproducibility. Within each about 1 to about 100 μm² dot, the immobilized oligonucleotides are essentially identical; that is, the oligonucleotides are all synthesized under the same conditions with the intent on creating identity and this identity is be achieved to a very high degree. While a variety of dot shapes can be used (e.g. square, rectangular, oblong, circular, trapezoidal, triangular, etc.), squares are commonly used because they divide and fill arrays efficiently while maximizing the array density.

In illustrative embodiments, the device includes a pattern comprising a plurality of dots. In one embodiment, the plurality of dots has at least one microscopic dimension. As used herein, a microscopic dimension is less than about 500 μm, less than about 400 μm, less than about 300 μm, less than about 200 μm, between about 1 μm and about 500 μm, or between about 10 μm and 250 μm. Essentially, the term microscopic indicates that the feature in at least one dimension is difficult to optically detect. Using a microscope, detection of an element having a microscopic dimension becomes possible. However, microscopic elements having microscopic dimensions are difficult to detect or analyze without magnification. In one embodiment, the dots have two microscopic dimensions. In one embodiment, the dots have dimensions such that the area is less than about 250,000 μm², less than about 160,000 μm², less than about 90,000 μm², less than about 40,000 μm², between about 36 μm² and about 250,000 μm², or between about 100 μm² and about 62,500 μm². In illustrative embodiments, a device according to the present disclosure includes at least one optically decipherable pattern that is a glyph rendered from a character selected from the Universal Character Set, defined by the International Standard ISO/IEC 10646. In one embodiment, the glyph is associated with a typeface, the typeface having a typographic size of between about 0.5 and about 216 points, between about 2 and about 144 points, or between about 3 and about 96 points, wherein one point is 1/72 of inch.

In illustrative embodiments, the device includes a dot including a plurality of binding molecules that specifically binds (e.g. is sufficiently complimentary) to a first target molecule and a second dot including a plurality of binding molecules that specifically binds a first control molecule. In one embodiment, the device includes a dot including a plurality immobilized oligonucleotides that are sufficiently complimentary to a first target oligonucleotide and a second dot includes including a plurality of immobilized oligonucleotides sufficiently complimentary to a first control molecule. Accordingly, two types of dots are described, a control dot containing detection molecules specific to control molecules and a target dot with immobilized detection molecules specific to a target sequence. In one embodiment, the device includes a multiplicity of control dots organized as a pattern. In another embodiment, the device includes a multiplicity of target dots organized as a pattern. Illustratively, these patterns are characters, for example glyphs of Unicode characters. In another embodiment, the device includes a plurality of different dot types (e.g. control, target 1, target 2, target 3, target 4, etc.), each dot type being present on the substrate surface in sufficient number and with organization so as to form a pattern. The number of dots sufficient for forming patterns is dependent on the size and shape of the dots and the size and shape of the pattern. The relationship between the exemplary detection molecules (immobilized nucleotides or peptides), the dots, and the patterns is shown in FIGS. 1-3.

In illustrative embodiments, the plurality of immobilized detection molecules are patterned on the substrate surface to form the at least one optically decipherable pattern rendered as at least one glyph, the at least one glyph comprising a plurality of dots (See FIG. 2 showing array of dots 20 and FIG. 3 showing how the dots make up Character p 38). Essentially, the dots are microscopic elements of the device that are not intended to be detected individually, but are meant to be collectively organized into positional and shape patterns which are optically decipherable. As described herein, the printing industry uses the term “dot” to refer to the size of a single deposition of ink onto a surface in the smallest increment. For example, a classic dot matrix printer uses rods striking a ribbon to apply ink and thus the dot size is related to the rod size. Dot matrix printers were known for resolutions in the range of 60 to 90 dots per inch (DPI). Inkjet printers spraying ink through tiny nozzles were typically capable of 300 to 600 DPI. In illustrative embodiments, a device according to the present disclosure includes dots patterned on the substrate surface between about 9 and about 4000 dots per inch, between about 150 and about 3000 dots per inch, or between about 300 and about 2000 dots per inch. In other embodiments, a device according to the present disclosure includes dots patterned on the substrate surface at greater than about 4000 dots per inch, about 3000 dots per inch, about 2000 dots per inch, about 1000 dots per inch, about 600 dots per inch, about 300 dots per inch, about 150 dots per inch, or about 72 dots per inch.

In illustrative embodiments, a device includes a high density microarray of dots. The present disclosure describes the use of dots as elements for forming larger patterns and characters. As in the printing industry, the resolution of the larger patterns is limited by the size of the individual dots from which it is formed. For example, a low resolution printing device such as a dot matrix printer will not be able to accurately convey the number of characters as an ink-jet printer. This problem is exacerbated as the size of the patterns decreases. As the device includes greater information density (e.g. greater number of characters), the resolution of the array becomes a significant factor. The resolution contributes to the accuracy, readability, and density of the device. In one embodiment, the device includes one or more resolution patterns. The resolution patterns include features that allow a user to assess whether the device and/or imaging equipment are sufficient for optically decipherable the patterns. This form of control provides another layer of quality control in performing an assay using an embodied device.

In illustrative embodiments, the device includes any suitable number of characters. In one embodiment, a character is specific to one target compound. Accordingly, the devices include features for detecting any suitable number of target compounds. For example, the device may include sufficient characters for detecting at least about 1, at least about 5, at least about 50, at least about 100, at least about 200, at least about 450, about 1, about 2, about 3, about 4, about 5, about 6, about 7, about 8, about 9, about 10, about 11, about 12, about 13, about 14, about 15, about 16, about 17, about 18, about 19, about 20, about 30, about 50, about 100, about 150, about 200, about 216, about 300, about 400, about 450, about 485, about 500, about 1000, about 10,000, about 25,000, about 41,420, about 50,000, about 72,000, no more than about 50, no more than about 500, no more than about 1,000, no more than about 10,000, no more than about 50,000, or no more than about 100,000 target compounds. Aspects of the device that influence suitability of a particular number of target compounds capable of being detected include size of the array, resolution of the detection molecule immobilization, immobilization chemistry accuracy/reproducibility, and desired readability.

An immobilized oligonucleotide can include any suitable nucleic acid sequence to detect the target nucleic acid. The immobilized oligonucleotide can be any length suitable for the detection of the target nucleic acid, including but not limited to lengths of at least about 20 bases, at least about 25 bases, at least about 35 bases, about 20 bases, about 25 bases, about 30 bases, about 35 bases, about 40 bases, about 45 bases, about 50 bases, about 55 bases, about 60 bases, about 70 bases, about 80 bases, about 90 bases, about 100 bases, no more than about 90 bases, or no more than about 100 bases. The minimum length of an immobilized oligonucleotide will, in some instances, be determined by the uniqueness of the immobilized oligonucleotide complement in the sample comprising the target nucleic acid.

In some instances, only a segment of the immobilized oligonucleotide is sufficiently complimentary to the target nucleic acid or one or more segments thereof. In some embodiments, sufficiently complimentary can be any suitable percentage complementarity, including but not limited to at least about 75%, at least about 90%, at least about 99%, about 75%, about 80%, about 85%, about 90%, about 95%, about 99%, about 99.9%, about 100%, or exact complementarity. In some embodiments, the immobilized oligonucleotide sequence is comprised of (1) one or more nucleic acid segments that each is sufficiently complimentary to one or more segments of the target nucleic acid (referred to as “the targeting segment(s)”) and (2) one or more nucleic acid segments that are not sufficiently complimentary to a segment of the target nucleic acid (referred to as “the non-targeting segment(s)”). The targeting segment of the immobilized oligonucleotide sequence can be any length suitable for the detection of the target nucleic acid, including but not limited to lengths of at least about 20 bases, at least about 30 bases, at least about 35 bases, about 20 bases, about 25 bases, about 30 bases, about 35 bases, about 40 bases, about 45 bases, about 50 bases, about 55 bases, about 60 bases, about 70 bases, about 80 bases, about 90 bases, about 100 bases, no more than about 90 bases, or no more than about 100 bases. The minimum length of a targeting segment will, in some instances, be determined by the uniqueness of the targeting segment complement in the test composition comprising the target nucleic acid.

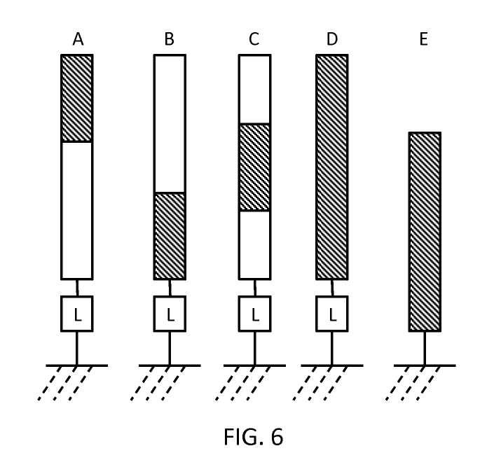

FIG. 6 provides an illustrative embodiment of the placement of the targeting segment. In some embodiments, the targeting segment of the immobilized oligonucleotide can be placed at any position within the immobilized oligonucleotide suitable for detection of the target nucleic acid, including but not limited to the free end of the immobilized oligonucleotide (i.e., the end of the immobilized oligonucleotide that is opposite the fixed end of the probe—See FIG. 6 Example A), the fixed end of the immobilized oligonucleotide (i.e., the end of the immobilized oligonucleotide that is fixed to the substrate—See FIG. 6 Example B), or between those two ends (i.e., non-targeting segments are on both sides of the targeting segment of the immobilized oligonucleotide sequence—See FIG. 6 Example C). In some embodiments, the entire length of the immobilized oligonucleotide may be complementary to a target oligonucleotide or a portion thereof (, the fixed end of the immobilized oligonucleotide (See FIG. 6 Example D). FIG. 6 further illustrates that the linker, shown as box marked L, may be optional in linking the immobilized oligonucleotide to the substrate surface (e.g. Example E).

In some embodiments, an immobilized oligonucleotide is attached to an attachment location on the substrate surface. The immobilized oligonucleotide can be attached to an attachment location on the substrate surface directly or through a linker. The linker can be any suitable molecule including but not limited to bivalent radicals of succinic acid, succinate. The attachment of the immobilized oligonucleotide to the substrate surface or to the linker can occur by any suitable chemical bond, including for example, by one or more covalent bonds, by one or more ionic bonds, by one or more hydrogen bonds, or combinations thereof. The point of attachment of the immobilized oligonucleotide to the substrate surface or to the linker can be at the nucleotide at the 3′ end of the immobilized oligonucleotide, at the nucleotide at the 5′ end of the immobilized oligonucleotide, or at a place on the immobilized oligonucleotide between the nucleotide at the 3′ end and the nucleotide at the 5′ end. The nucleotide point of attachment can, in some instances, be the nucleotide base, the nucleotide sugar, or the nucleotide phosphate. In some embodiments, the fraction of immobilized oligonucleotides attached to the substrate surface at the 3′ end of the immobilized oligonucleotide can be at least about 75%, at least about 90%, about 75%, about 80%, about 90%, about 99%, about 99.9%, about 100%, all of the immobilized oligonucleotides, no more than 99%, or no more than 99.9%. In one embodiment, the oligonucleotides are not attached to the substrate through a drying process (e.g. not physically adhered oligonucleotides). One aspect of the present disclosure is that digital optical chemistry approaches to synthesizing arrays provides immobilized oligonucleotides with favorable orientation, availability, and control than drying approaches (e.g. dot blotting or microspotting). In another embodiment, the patterns that are formed with bound oligonucleotides are superior to those formed through drying procedures in terms of resolution, accuracy, control, and reproducibility.

The dots are characterized in that they include a substantially identical plurality of immobilized oligonucleotides. In one embodiment, each dot includes a single binding moiety. In one embodiment, at least about 75%, 90%, 95%, or 99% of the plurality of immobilized oligonucleotides bound to the substrate surface within each of the plurality of dots have identical nucleic acid sequences. While the sequences of the immobilized oligonucleotides within one dot are substantially identical, it was discovered that the sequences of the immobilized oligonucleotides within dots that form one pattern are advantageously not identical.

In illustrative embodiments, the device includes a plurality of immobilized oligonucleotides tiled across a target or control nucleotide sequence within one pattern on the substrate surface. It was discovered that tiling immobilized oligonucleotide sequences across a target or a control sequence provides advantages; particularly, the device is capable of binding a greater portion of the target or control oligonucleotide. As described herein, within each dot the immobilized oligonucleotides may be substantially identical. Furthermore, across each pattern, glyph, or character, the immobilized oligonucleotides may all be complimentary to a given target sequence. One aspect of the present disclosure is that the immobilized oligonucleotides are often substantially shorter in length than the target oligonucleotides in the sample. As such, the entire length of the immobilized oligonucleotide can be designed to hybridize to a portion of the target oligonucleotide, but it may be impractical to design an immobilized oligonucleotide that hybridizes to the entire length of the molecular target (e.g. the target oligonucleotide). Using identical nucleic acid sequences in each of the dots that make up a particular character would only query a specific portion of the molecular target. This is problematic where the target oligonucleotide is very long and broken into many shorter haptenated sequences for analysis. In this case, only a portion (possibly a very small portion) of the shorter haptenated sequences will bind to the immobilized oligonucleotides.

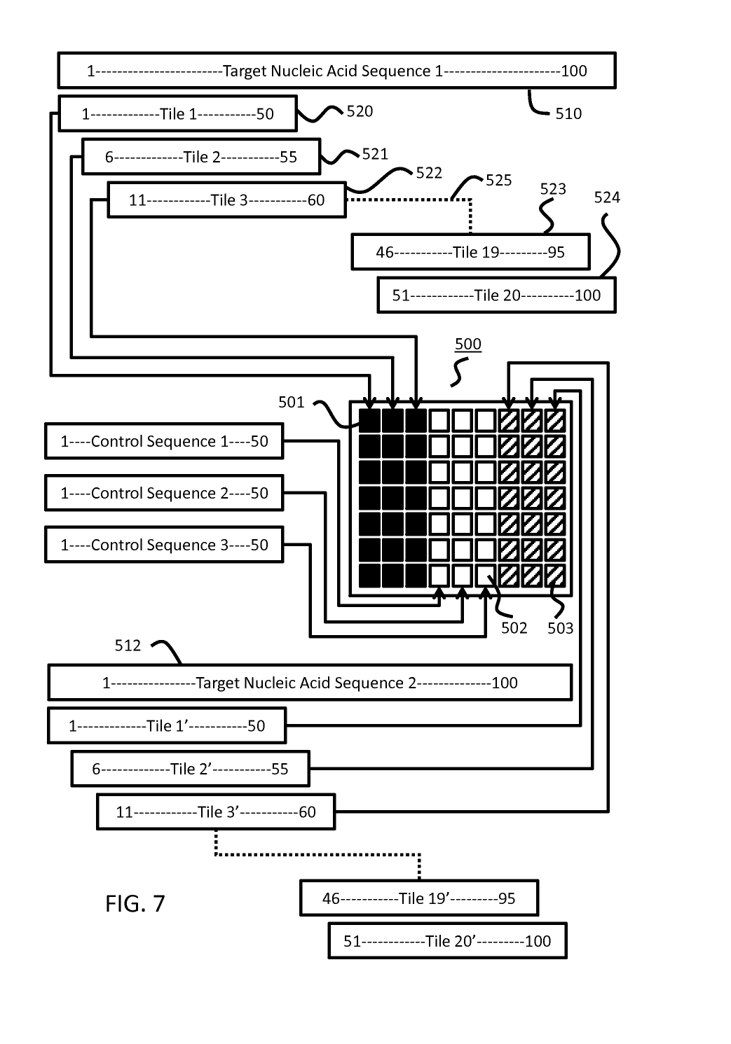

Referring now to FIG. 7, shown is a schematic of a method of using a device on an analyzer to generate a decipherable pattern. Shown centrally is a portion 500 of an exemplary microscopic array having a pattern of 7 rows and 9 columns of dots. The array extends for substantial lengths in both dimensions, portion 50 is shown merely as an example. Portion 500 includes three distinct types of immobilized oligonucleotide sequences that are each shown immobilized according to a plurality of dots. For example, solid squares 501 represent those dots associated with Target Nucleic Acid Sequence 1 510, hollow squares 502 represent those dots associated with Control Sequences 511, and cross-hatched squares 503 represent those dots associated with Target Nucleic Acid Sequence 2 512. The boxes 520 to 524 show a manner in which Target Nucleic Acid Sequence 1 510 can be tiled across the microscopic array. Dotted line 525 conveys that Tile 4 through Tile 18 repeat according to the manner show for Tiles 1-3 and Tiles 19-20. As shown, Target Nucleic Acid Sequence 1 510 is tiled by 5 bp for each dot. After the entire length of Target Nucleic Acid Sequence 1 510 has been tiled across, the tiling can be repeated. As shown, the pattern associated with Target Nucleic Acid Sequence 1 510 would include dots with one of 20 pre-determined sequences. Each of the 20 sequences are derived from Target Nucleic Acid Sequence 1 510, thus would bind a portion thereof. However, each of the 20 sequences would bind to a different portion of Target Nucleic Acid Sequence 1 510. An alike approach is shown for Target Nucleic Acid Sequence 2 512. While FIG. 7 is particular to immobilized nucleic acids, the approach described therein may also be applied to nucleic acid mimics, peptides, carbohydrates, etc.

In illustrative embodiments, Control Sequences 511 are not tiled. Instead, these sequences can be individually selected from either a positive or negative sequence population. For example, if Control Sequences 511 are positive controls for the presence of human DNA, the sequences may be selected from a population of known human DNA repeat sequences. If Control Sequences 511 are negative controls, the sequences may be selected from a population of uniquely distinct sequences (e.g. U.S. Published Application No. 2012/0070862, herein incorporated by reference in its entirety for disclosure related to uniquely distinct sequences and the generation thereof). While control sequences can be tiled, it may not be necessary or appropriate depending on the objective of the control. Additionally, the population of possible control sequences may be sufficiently large that tiling has the effect of decreasing the range of sequence diversity. It was discovered that tiling across target DNA sequences within characters enhances the detection limit as a higher percentage of the target nucleic acids bind to the device. This discovery was particularly prescient to large targets which are haptenated through a nicking process.

For example, a target sequence is selected that is 500 bp in length and the immobilized oligonucleotides are identical 50-mers complementary to base pairs 1-50 of the target sequence. Nicking the 500 bp target sequence to form shorter haptenated sequences may illustratively generate in ten 50 bp resultant target oligonucleotides. If the dots making up a single character are not tiled, only 10% of the resultant target oligonucleotides would bind to the immobilized oligonucleotide since those immobilized oligonucleotides are only complementary to 10% of the target sequence length. Detecting only 10% of the target decreases sensitivity and potentially introduces inaccuracies if there is a biased nick site within the sequence selected for the immobilized oligonucleotide. Upon tiling sequences across the dots as in certain illustrative embodiments, the device includes characters comprised of multiple dots, and the dots tile across a first nucleotide sequence by a suitable number of base pairs. In one embodiment, the dots tile across a first nucleotide sequence by a number nearly equal to or less than the number of base pairs in the immobilized oligonucleotides. In another embodiment, the dots tile across between 1 and 50 base pairs, 2 and 25 base pairs, or 5 and 10 base pairs.

In some embodiments, a suitable number of target regions on a substrate surface are used as positive controls, negative controls, or both. The number of positive control target regions can be any suitable number of positive control regions, including but not limited to 1, 2, 3, 4, 5, 6, 7, 8, 9, 10, 15, 20, 25, 30, 35, 40, 45, 48, 50, 55, 60, 61, 65, 75, 85, 95, 100, 108, 150, 200, 208, 250, 300, 350, 400, 450, 485, or 500. The number of negative control target regions can be any suitable number of negative control regions, including but not limited to 1, 2, 3, 4, 5, 6, 7, 8, 9, 10, 15, 20, 25, 30, 35, 40, 45, 48, 50, 55, 60, 61, 65, 75, 85, 95, 100, 108, 150, 200, 208, 250, 300, 350, 400, 450, 485, or 500. For example, a positive control can include, but is not limited to, plasmid backbone sequences if one or more target nucleic acids is prepared using a plasmid. For example, a negative control target region can include, but is not limited to, an immobilized oligonucleotide sequence that only provides non-specific binding, an immobilized oligonucleotide sequence that is a randomized sequence (e.g., a sequence that has little to no homology to the sequences of any of the target nucleic acid sequences), an immobilized oligonucleotide sequence that is a sequence from one or more organisms unrelated to the organism(s) from which the target nucleic acids are derived. Appropriate sequences for negative controls may include uniquely distinct sequences (e.g. U.S. Published Application No. 2012/0070862). In some instances, a target region will include an attachment location that does not have anything attached at the location. For example, the substrate surface may be unmodified in that location. In another embodiment, the substrate surface may be modified with only the linker. In other embodiments, the device includes controls that are patterned as one or more characters so that the control is optically decipherable. In yet other embodiments, a device includes a positive control that is an oligonucleotide having a uniquely human sequence to verify successful binding of a human DNA. In another embodiment, a positive control is an oligonucleotide having a sequence unique to the Y chromosome to establish gender of the source genetic material. In another embodiment, a positive control is specific to bacterial protein or gene common to the classification, for example, where the target is a strain specific marker.

In further embodiments, a device includes a control binding compound that is incapable of hydrogen bonding but allows for other DNA binding mimicry such as base stacking or intercalation. In yet other embodiments, a control includes non-biological amino acids that can absorb background signal to calibrate for non-specific detection chemistry interaction, which otherwise would not create structure for proteins to bind to specifically. In yet other embodiments, a control includes iteratively truncated versions of the detection molecules. Truncated detection molecules would enable to test stringency and/or other binding characteristics. For example, when a signal is observed from negative truncated control, it may indicate that assay conditions failed to meet stringency requirements. In still other embodiments, a device could include hapten control features. In one embodiment, a hapten could be bound to device prior to testing for verification that the detection chemistry is effective.

In illustrative embodiments, a method of analyzing a sample using an array device comprises contacting the array device with a sample solution, contacting the array device with an antibody solution, the antibody solution comprising an antibody conjugated to an enzyme, contacting the array device with a chromogenic detection solution, the chromogenic detection solution comprising a compound that interacts with the enzyme to deposit a chromogenic substance on the array device to form at least one optically decipherable pattern, and deciphering the optical pattern. Fluids can be contacted with the device by any means. For example, sample and/or detection reagents may be contacted with the device by pipetting, spraying, misting, painting, squeegeeing, or any other suitable means. The dispensing of reagents and other fluids is desirably automated. Thus, the selection of the fluid to be dispensed and the amount and manner in which the fluid is dispensed can be computer controlled. U.S. Patent Publication 2008/0102006, the entire disclosure of which is incorporated herein by reference, describes robotic fluid dispensers that are operated and controlled by microprocessors.

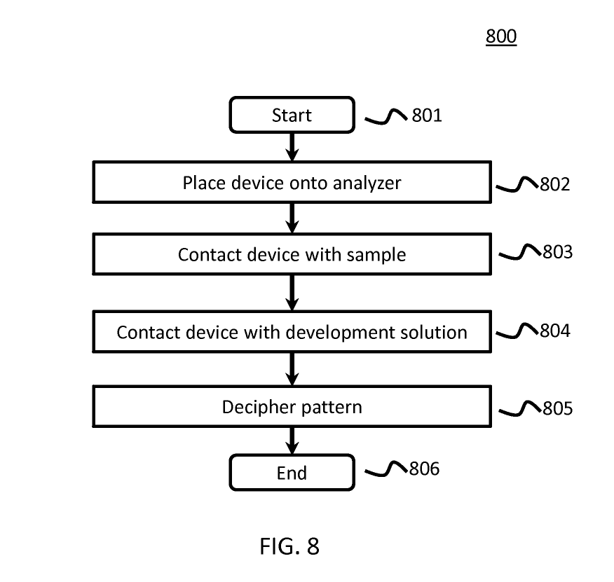

In illustrative embodiments, a method according to the present disclosure uses an automated instrument. In one embodiment, the steps of contacting the array device with the chromogenic detection solution, contacting the array device with the antibody solution, and/or contacting the array device with a sample solution, are selected to be performed by an automated instrument. In one embodiment, the array device comprises a plurality of immobilized oligonucleotides patterned on the array device and contacting the array device with the sample solution includes hybridizing a plurality of target oligonucleotides with the plurality of immobilized oligonucleotides. In another embodiment, the plurality of target oligonucleotides are hapten-labeled oligonucleotides and contacting the array device with the sample solution includes labeling the plurality of immobilized oligonucleotides. In another embodiment, the antibody solution includes an anti-hapten antibody and contacting the array device with the antibody solution includes binding the anti-hapten antibody to the hapten-labeled oligonucleotide. Particular methods require that a sample or detection reagent be in contact with the device for a specified amount of time. Accordingly, one aspect of automation is the implementation of precisely controlled incubation steps performed at pre-determined temperatures for pre-determined periods of time. Referring now to FIG. 8, shown is a schematic of an illustrative method of using a device on an analyzer to generate a decipherable pattern. An automated method 800 includes a start 801, a step of placing a device according to the present disclosure onto an instrument 802, contacting the device with sample 803, contacting the device with a development solution(s) 804, deciphering one or more patterns 805, and an end 806.

In illustrative embodiments, a device according to the present disclosure uses or is designed for use with chromogenic detection reagents. There are numerous advantages to the manner in which devices according to the present disclosure are compatible with chromogenic detection reagents. One advantage is that chromogenic detection reagents are readily available for use with automated slide stainers. As used herein, the term automated refers to a method where one or more steps are executed by substantially mechanical, electro-mechanical, computer, and/or electronically controlled systems. It does not exclude some human intervention steps such as loading samples on slides and/or manually performing one or more of the features or steps described herein. As such, many benefits attributable to automation can be made benefits of the present devices. For example, automated slide staining is faster, requires less resources (e.g. cheaper, less reagents, less labor), and more reproducible than manual procedures. In some embodiments, methods according to the present disclosure include automated chromogenic detection.

Automated systems typically are at least partially, if not substantially entirely, under computer control. Because automated systems typically are at least partially computer controlled, certain embodiments of the present disclosure also concern one or more tangible computer-readable media that stores computer-executable instructions for causing a computer to perform disclosed embodiments of the method. Particular disclosed embodiments concern a computer-controlled, bar code driven, staining instrument that automatically applies chemical and biological reagents to samples, such as tissue and/or cell samples, that are mounted or affixed to a slide. More than one slide may be used, with particular embodiments using from about 1 to about 50 slides; in other embodiments from about 1 to about 20 slides.

The disclosed embodiments may be used with any of various automated staining systems, particularly those provided by Ventana Medical Systems, Inc., including the Benchmark XT, Benchmark Ultra, and Discovery systems. Exemplary systems are disclosed in U.S. Pat. No. 6,352,861, U.S. Pat. No. 5,654,200, U.S. Pat. No. 6,582,962, U.S. Pat. No. 6,296,809, and U.S. Pat. No. 5,595,707, all of which are incorporated herein by reference. The following description exemplifies a suitable embodiment of an automated method and system. Additional information concerning automated systems and methods also can be found in PCT/US2009/067042 published as WO/2010/080287, which is incorporated herein by reference. Chromogenic detection facilitates visual unaided deciphering of patterns on the device. However, chromogenic detection is also compatible with brightfield imaging. Brightfield imaging involves transmitting white light in the visible spectrum through the chromogens. The chromogen absorbs and/or reflects light of certain wavelengths and transmits other wavelengths. The reflected and/or transmitted light exhibits a color characteristic of the particular chromogen used.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed. Embodiments of automated systems may perform all or any subset of, processing steps of processing, staining and coverslipping of devices according to the present disclosure.

Another advantage of chromogenic detection reagents is that the chromogenic signals are optically decipherable patterns. In illustrative embodiments, chromogenic signals are visually decipherable patterns. Several analytical methods currently available enable extraordinary sensitivity, even single-molecule detection; however, these approaches and these levels of detection require sophisticated instrumentation. In illustrative embodiments, a device according to the present disclosure does not require sophisticated instrumentation. Instead, an illustrative device includes an optically decipherable pattern. In one embodiment, the decipherable pattern includes a shape pattern. In another embodiment, the decipherable pattern includes a shape pattern and a positional pattern. While even signals observed performing single molecule detection may be called optical signals (e.g. SERS, fluorescence, luminescence), those signals lack patterning as described herein. In particular, the patterns referred to herein carry intrinsic semantic meaning or a code therefore. Chromogenic detection reagents are well-suited for visual identification and deciphering. Chromogenic signals are not transient like many fluorophores and luminophores which may degrade or become passivated over time. Furthermore, chromogenic signals do not blink or otherwise significantly fluctuate in intensity. Rather, the stability of chromogenic species can be relied on to provide an enduring record for the results of a particular analysis.

In illustrative embodiments, described is a device for memorializing a hybridization event comprising a substrate with at least one substrate surface, a plurality of immobilized detection molecules bound to the substrate surface, a plurality of target molecules bound or hybridized to the plurality of immobilized detection molecules, the plurality of target molecules including labels, and a plurality of stable chromogenic depositions localized proximally to the plurality of immobilized detection molecules, wherein the labels direct the plurality of stable depositions to form an optically decipherable pattern on the substrate, the optically decipherable pattern memorializing the hybridization or binding event. In one embodiment, the labels include a hapten. In another embodiment, the labels include a hapten bound to an enzyme-conjugated anti-hapten antibody. In another embodiment, the enzyme-conjugated anti-hapten antibody includes an HRP (horse radish peroxidase) or AP (alkaline phosphatase). In one embodiment, the pluralities of stable chromogenic depositions are immunohistochemical stains. In yet another embodiment, the stable chromogenic depositions are selected from silver, nickel, DAB (diaminobenzidine), fast red (4-chloro-2-methylbenzenediazonium/3-hydroxy-2-naphthoic acid 2,4-dimethylanilide phosphate), fast blue (o-dianisidine bis(diazotized) zinc double salt), and BCIP/NBT (5-Bromo-4-chloro-3-indolyl phosphate/nitro blue tetrazolium chloride). In one embodiment, the plurality of target oligonucleotides includes a cocktail of at least two uniquely specific DNA probes. In another embodiment, the optically decipherable pattern comprises a plurality of microscopic array features having at least one dimension less than about 200 μm. In another embodiment, the optically decipherable pattern includes at least one feature having a length of between about 200 μm and 75 millimeters. The device enables memorialization of the hybridization event because the stable depositions on the device provide an archival record that a specific binding event occurred. The archival record is stable for multiple examinations and storage and is additionally not reliant on sophisticated imaging analysis and software which may or may not be available after significant storage periods.

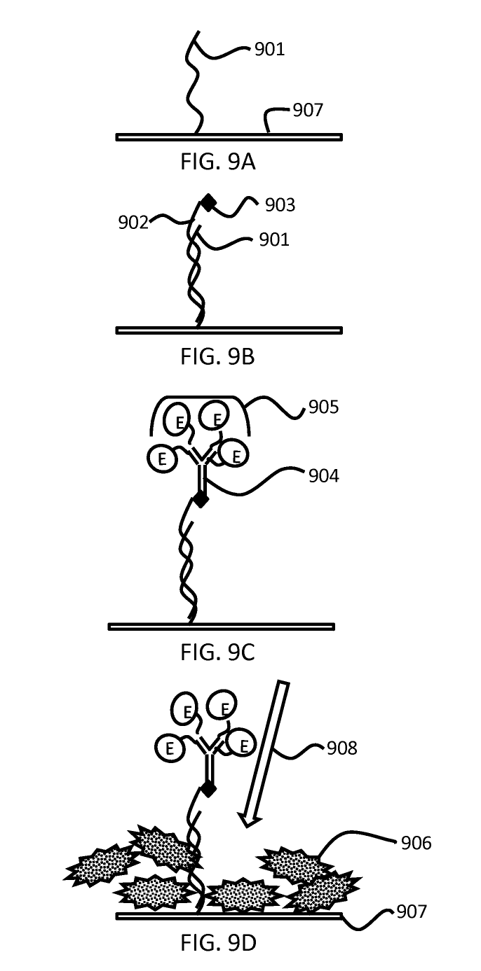

Referring now to FIG. 9(A-D), shown are diagrammatic representations of a progression of a method according to one embodiment of the present invention in which (A) shows an immobilized oligonucleotide 901 on a substrate surface 907, (B) shows a complementary oligonucleotide 902 hybridized to immobilized oligonucleotide 901, oligonucleotide 902 being haptenated with a first hapten 903, (C) shows an anti-hapten antibody 904 conjugated with enzyme 905, and (D) shows an enzymatically deposited chromogenic substance 906 deposited on substrate surface 907. An arrow 908 indicates that soluble chromogen is converted into enzymatically deposited chromogenic substance 906 by enzyme 905.

The approach shown in FIG. 9(A-D) is an “AB” detection approach, where the primary antibodies are detected via anti-species recognition between the binding domain of the secondary Ab and Fc domain of the primary antibody. In exemplary embodiments, detection is realized through anti-species antibodies conjugated with multiple enzymes (e.g. horse radish peroxidase (HRP) and alkaline phosphatase (AP)). This enzyme-antibody conjugate is referred to as an HRP or AP multimer in light of the multiplicity (e.g. shown as four in FIG. 9) of enzymes conjugated to each antibody. Multimer technologies are described in U.S. Published Application. No. 2010/0136652 which is hereby incorporated by reference in its entirety for disclosure related to antibody conjugates. AB detection approaches may be faster than so called “ABC” approaches because of the reduced number of steps. This type of detection chemistry is currently marketed by Ventana Medical Systems Inc. as Universal DAB detection kit (P/N 760-500), Universal AP Red detection kit (P/N 760-501), Red ISH DIG detection kit (P/N 760-505), and SISH DNP detection kit (P/N 760-098).

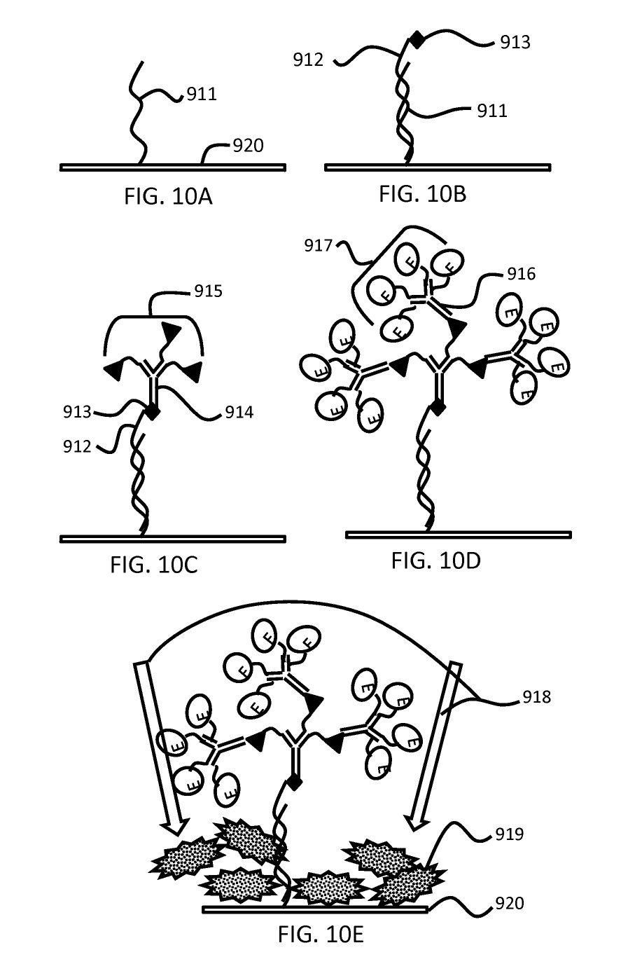

Referring now to FIG. 10(A-E), shown are diagrammatic representations of a progression of a method according to one embodiment of the present invention in which (A) shows an immobilized oligonucleotide 911 on a substrate surface 920, (B) shows a complementary oligonucleotide 912 hybridized to immobilized oligonucleotide 911, oligonucleotide 912 being haptenated with a first hapten 913, (C) shows an anti-hapten antibody conjugate 914 with a secondary hapten 915 bound to hapten 913, (D) shows an anti-hapten antibody 916 conjugated with enzyme 917 bound to hapten 915, and (E) shows an enzymatically deposited chromogenic substance 919 deposited on substrate surface 920. Arrows 918 indicate that soluble chromogen is converted into enzymatically deposited chromogenic substance 919 by enzyme 917.

The approach shown in FIG. 10(A-E) is an “ABC” approach using haptenated secondary anti-species antibodies followed by anti-hapten antibody enzyme conjugate multimers to detect the primary antibodies. In illustrative embodiments, the approach uses non-endogenous haptens (e.g. not biotin, reference is made to U.S. application Ser. No. 12/660,017, published as US 2010/0184087, which is hereby incorporated by reference in its entirety for disclosure related to detection chemistries). In illustrative embodiments, tyramide signal amplification may be used with this approach to further increase the sensitivity and dynamic range of the detection (see PCT/US2011/042849, published as WO2012003476, which is hereby incorporated by reference in its entirety for disclosure related to detection chemistries). While both FIGS. 9 and 10 show an immobilized oligonucleotide as the immobilized detection molecule and a haptenated oligonucleotide as the target molecule, the immobilized detection molecule could be any suitable detection molecule including immobilized peptides, haptens, proteins, antibodies, chelators, ligands, glycosylated proteins having carbohydrates thereon, haptenated peptides, peptides having phosphorylated tyrosines or combinations of the same. Those skilled in the art will appreciate that the nature of the molecular target dictates the approach to initial detection. FIGS. 9 and 10 show illustrative approaches for staining or colorimetrically developing the initial detection event.

Any suitable enzyme/enzyme substrate system can be used for the disclosed automated method. Working embodiments provided herein as examples use alkaline phosphatase and horseradish peroxidase. If the enzyme is alkaline phosphatase, one suitable substrate is nitro blue tetrazolium chloride/(5-bromo-4-chloro-1H-indol-3-yl)dihydrogen phosphate (NBT/BCIP). If the enzyme is horseradish peroxidase, then one suitable substrate is diaminobenzidine (DAB), fast red or fast blue. Numerous other enzyme-substrate combinations are known to those skilled in the art. A general review of enzyme-substrate combination is described U.S. Pat. Nos. 4,275,149 and 4,318,980. In some embodiments, the enzyme is a peroxidase, such as horseradish peroxidase or glutathione peroxidase or an oxidoreductase. Thus, suitable conditions are selected for enzyme reaction such as a salt concentration and pH that enable the enzyme to perform its desired function, for example to convert the substrate to a detectable moiety that is deposited on the tissue sample at the site of the target molecule. The reaction is performed at a temperature that is suitable for the enzyme. For example, if the enzyme is horseradish peroxidase, the reaction may be performed between about room temperature and about 35-40° C.

In illustrative embodiments, the device includes one or more chromogens or the method of using the device includes one or more distinct chromogenic detection steps. In one embodiment, the device includes two chromogens or the method of using the device includes two distinct chromogenic detection steps. In another embodiment, the device includes three chromogens or the method of using the device includes three distinct chromogenic detection steps. In yet another embodiment, the device includes four chromogens or the method of using the device includes four distinct chromogenic detection steps.

Certain aspects, or all, of the disclosed embodiments can be automated, and facilitated by computer analysis and/or image analysis system. In some embodiments, an image of the device (or of any target area of the device) can be acquired by photography (e.g., using digital or film photography) or by scanning (e.g., by manual or automatic scanning). In still further embodiments, the acquired image can be converted into a computer readable image (e.g., by digitizing the acquired image). The computer readable image can, in some embodiments, be analyzed by a computer, by a human, or using combinations thereof. In certain instances, a computer will analyze the computer readable image and then the computer will prompt a human to answer questions regarding the image, thereby further analyzing the computer readable image. In some applications, optical character recognition (OCR) is used to decipher the patterns. In some embodiments, light microscopy is utilized for image analysis. Certain disclosed embodiments involve acquiring digital images. This can be done by coupling a digital camera to a microscope, using a camera alone, or using a scanning device. Digital images obtained of exemplary devices may be analyzed using image analysis software. The image analysis software can analyze shape and/or color. Color can be measured in several different ways. For example, color can be measured as red, blue, yellow, and green values; hue, saturation, and intensity values; and/or by measuring a specific wavelength or range of wavelengths using a spectral imaging camera.

In illustrative embodiments, deciphering the optical pattern includes using automated identification and data capture, wherein a computer is used to relate the at least one pattern to a meaning associated with the plurality of immobilized detection molecules. In one embodiment, the at least one optically decipherable pattern is an information code. In another embodiment, the information code is an optical machine-readable representation of data. In another embodiment, the information code is a 1-dimensional data code or a 2-dimensional data code. For example, the information code could be a bar code or a QR code. According to these embodiments, the devices can be computer readable, (i.e. in that a computer can be programmed to correlate a particular address on the array with information about the sample). In one embodiment, the device can include both visually decipherable patterns and machine readable patterns. In another embodiment, the device includes redundant visually decipherable patterns and machine readable patterns so that a use can visually decipher the device and have a computer verify the reading, or vice versa.

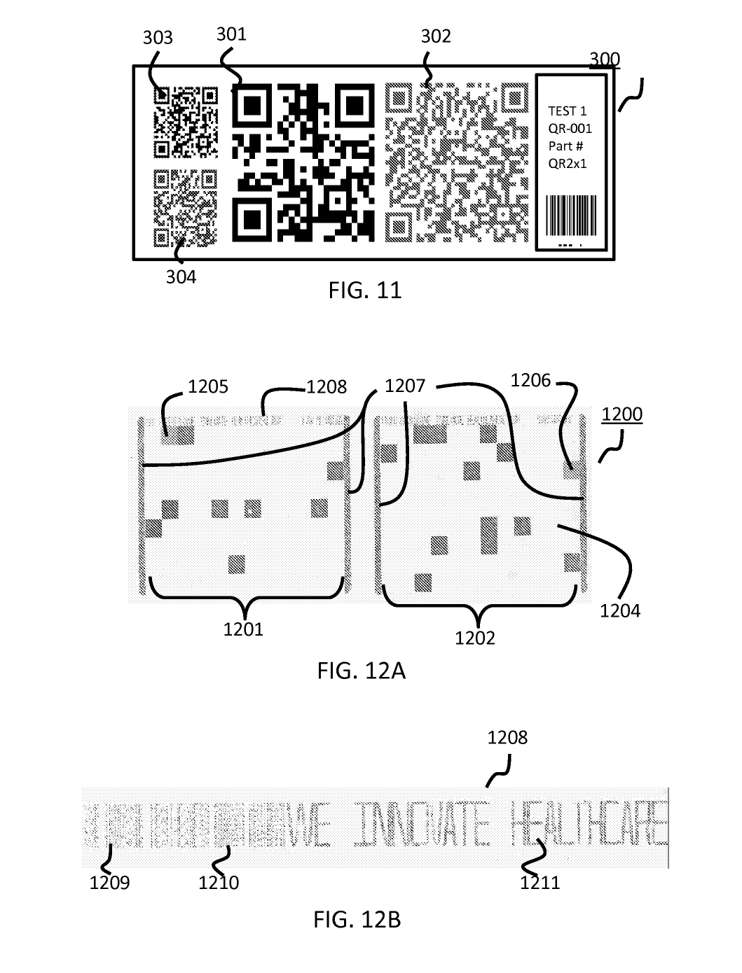

Referring now to FIG. 11, shown is a top plan view of a device 300 according to an illustrative embodiment of the present invention showing four optically decipherable patterns 301, 302, 303, and 304. Each of the patterns are made in accordance with the technical specifications for a QR Code, set forth in the ISO-18004 standard. The optically decipherable patterns can be deciphered by a camera-enabled computing device (e.g. computer, mobile phone, tablet) with the appropriate software to link the computing device to a predetermined website, data, or text. Computing devices and software useful for this task are now widespread. The optically decipherable pattern 301 can be deciphered by a camera-enabled computing device to display a website. The optically decipherable patterns 302, 303, and 304 can be deciphered by a camera-enabled computing device to display a text message. In case of the website link or the text message, information regarding the binding events that occurred on the device can be conveyed to the user. In illustrative embodiments, a device according to the present disclosure includes patterns readable by a camera-enabled handheld computing device. The size of the data modules on a QR Code is limited by the resolution of the optics of the camera-enabled computing devices. For instance, the average camera-enabled computing device now has a resolution such that each data module should be at least 0.4 mm by 0.4 mm (400 μm by 400 m).

### EXAMPLES

### Example 1

### DNA Probe Confirmation Test

Several studies were performed to evaluate illustrative devices for multiplexed visual identification of a mixed population of DNA probes. The study confirmed the device's utility for assessing a composition including a mixture of DNA probes.

The ability of the device to undergo readable chromogenic staining was demonstrated. Referring now to FIG. 12(A-B), shown is an exemplary device 1200 memorializing the binding of various oligonucleotide targets. Device 1200 includes two identification regions 1201 and 1202. Identification regions 1201 and 1202 include 9 row by 12 column arrays of rectangles (Unicode 25AC, Black Rectangle). FIG. 12A shows rectangles chromogenically developed in both red or black (while the Unicode character is named black rectangle, there is no requirement that the character be black). In this case, the black signal indicates that a DNP (2,4-dinitrophenol) labeled probes were hybridized to immobilized oligonucleotides within the pattern shapes to effectively label the immobilized oligonucleotides with a DNP hapten. The hapten was chromogenically developed using commercially-available detection reagents on an automated instrument (see Table 1). Similarly, a DIG (digoxigenin) labeled probe was hybridized to immobilized oligonucleotide within the other characters (see rectangles 1205 and 1206) to effectively label the immobilized oligonucleotide with a DIG hapten. Those regions that do not exhibit any color (e.g. region 1204 of FIG. 12A) are indicative of no labeling; thus, no binding event occurred between a detected hapten-labeled oligonucleotide and the immobilized oligonucleotide in those regions. Device 1200 was constructed with immobilized oligonucleotides covering the entire region between and including heavy verticals 1207. The lack of color in the remainder of the identification regions can be attributed to the test solution being devoid of hapten-labeled oligonucleotides sufficiently complimentary to the immobilized oligonucleotides in those regions.

The materials, parts, and instruments used to perform the analysis memorialized in FIG. 12(A-B) are shown in Table 1. The following materials available from Ventana Medical Systems, Inc.

TABLE 1
Materials for DNA Probe Confirmation Test

| | Part Number |
|---|---|
| BENCHMARK ULTRA | N750-BMKU-FS |
| Automated Instrument | |
| HYBREADY | 780-4409 |
| EZ Prep (10X) | 950-102 |
| Reaction Buffer (10×) | 950-300 |
| High Temperature Liquid | 650-010 |
| CoverSlip | |
| SSC | 950-110 |
| Red ISH DIG Detection Kit | 760-505 |
| SISH DNP Detection Kit | 760-098 |
| Silver Wash II | 780-003 |

The materials were used according to the following protocol on the automated instrument: 1 Enable Mixers; 2 Rinse Slide With EZ Prep; 3 Adjust Slide Volume With EZ Prep; 4 Apply Coverslip; 5 Rinse Slide With EZ Prep; 6 Apply 150 ul of EZ Prep; 7 Apply Coverslip; 8 Rinse Slide With SSC; 9 Apply 300 ul of SSC; 10 Apply Coverslip; 11 Rinse Slide With SSC; 12 Apply 300 ul of SSC; 13 Apply Coverslip; 14 Warmup Slide to 36 Deg C.; 15 Rinse Slide With SSC; 16 Adjust Slide Volume With SSC; 17 Warmup Slide to 42 Deg C.; 18 Apply 150 ul of SSC; 19 Apply CC Coverslip Long; 20 Apply Two Drops of HYB RDY SOL, and Incubate for 4 Minutes; 21 Apply Two Drops of HYB RDY SOL, and Incubate for 4 Minutes; 22 Apply One Drop of [Haptenated Test Oligonucleotide], No Coverslip and Incubate for 4 Minutes; 23 Warmup Slide to [35 Deg C] from All Temperatures (Hybridization); 24 Apply CC Coverslip Short No BB; 25 [hybridization time]; 26 Incubate for [6 Hours]; 27 Disable Slide Heater; 28 Rinse Slide With SSC; 29 Apply 450 ul of SSC; 30 Apply Coverslip; 31 Incubate for 8 Minutes; 32 Rinse Slide With SSC; 33 Apply 450 ul of SSC; 34 Apply Coverslip; 35 Incubate for 8 Minutes; 36 Rinse Slide With SSC; 37 Apply 450 ul of SSC; 38 Apply Coverslip; 39 Incubate for 8 Minutes; 40 Rinse Slide With SSC; 41 Apply 450 ul of SSC; 42 Apply Coverslip; 43 Pause Point (Landing Zone); 44 Warmup Slide to 36 Deg C., and Incubate for 4 Minutes; 45 Rinse Slide With Reaction Buffer; 46 Apply One Drop of SIL ISH DNP RAB, Apply Coverslip, and Incubate for 24 Minutes; 47 Rinse Slide With Reaction Buffer; 48 Adjust Slide Volume With Reaction Buffer; 49 Apply Coverslip; 50 Rinse Slide With Reaction Buffer; 51 Adjust Slide Volume With Reaction Buffer; 52 Apply Coverslip; 53 Rinse Slide With Reaction Buffer; 54 Apply One Drop of SIL ISH DNP HRP, Apply Coverslip, and Incubate for 0 Hr 36 Min; 55 Rinse Slide With Reaction Buffer; 56 Adjust Slide Volume With Reaction Buffer; 57 Apply Coverslip; 58 Rinse Slide With Reaction Buffer; 59 Adjust Slide Volume With Reaction Buffer; 60 Apply Coverslip; 61 Disable Slide Heater; 62 Rinse Slide With Reaction Buffer; 63 Adjust Slide Volume With Reaction Buffer; 64 Apply Coverslip; 65 Rinse Slide With Reaction Buffer; 66 Jet Drain With Reaction Buffer; 67 Apply One Drop of SIL ISH DNP CHRA, Apply Coverslip, and Incubate for 4 Minutes; 68 Rinse Slide With Reaction Buffer; 69 Adjust Slide Volume With Reaction Buffer; 70 Apply Coverslip; 71 Rinse Slide With Reaction Buffer; 72 Adjust Slide Volume With Reaction Buffer; 73 Apply Coverslip; 74 Rinse Slide With Option; 75 Adjust Slide Volume With Option; 76 Apply Coverslip; 77 Rinse Slide With Option; 78 Apply 150 ul of Option; 79 Apply 50 ul of Reaction Buffer; 80 Apply Coverslip; 81 Apply One Drop of SIL ISH DNP CHRA, and Incubate for 4 Minutes; 82 Apply One Drop of SIL ISH DNP CHRB, and Incubate for 4 Minutes; 83 [Silver Chromogen]; 84 Apply One Drop of SIL ISH DNP CHRC, and Incubate for [0 Hr 20 Min]; 85 Rinse Slide With Option; 86 Adjust Slide Volume With Option; 87 Apply Coverslip; 88 Rinse Slide With Reaction Buffer; 89 Adjust Slide Volume With Reaction Buffer; 90 Apply Coverslip; 91 Pause Point (Landing Zone); 92 Warmup Slide to 36 Deg C.; 93 Rinse Slide With Reaction Buffer; 94 Adjust Slide Volume With Reaction Buffer; 95 Apply Coverslip; 96 Rinse Slide With Reaction Buffer; 97 Apply One Drop of RED ISH DIG MAB, Apply Coverslip, and Incubate for 24 Minutes; 98 Rinse Slide With Reaction Buffer; 99 Adjust Slide Volume With Reaction Buffer; 100 Apply Coverslip; 101 Rinse Slide With Reaction Buffer; 102 Adjust Slide Volume With Reaction Buffer; 103 Apply Coverslip; 104 Rinse Slide With Reaction Buffer; 105 Apply One Drop of RED ISH DIG AP, Apply Coverslip, and Incubate for 24 Minutes; 106 Rinse Slide With Reaction Buffer; 107 Adjust Slide Volume With Reaction Buffer; 108 Apply Coverslip; 109 Rinse Slide With Reaction Buffer; 110 Adjust Slide Volume With Reaction Buffer; 111 Apply Coverslip; 112 Rinse Slide With Reaction Buffer; 113 Adjust Slide Volume With Reaction Buffer; 114 Apply Coverslip; 115 Disable Slide Heater; 116 Rinse Slide With Reaction Buffer; 117 Adjust Slide Volume With Reaction Buffer; 118 [Fast Red Cycle 1]; 119 Apply Two Drops of RED ISH DIG PHE, Apply Coverslip, and Incubate for 8 Minutes; 120 Apply One Drop of RED ISH DIG NAP, and Incubate for 4 Minutes; 121 Apply One Drop of RED ISH DIG FR, and Incubate for 4 Minutes; 122 [Red Chromogen]; 123 Apply One Drop of RED ISH DIG FR, and Incubate for [0 Hr 24 Min]; 124 [Fast Red Cycle 2]; 125 Rinse Slide With Reaction Buffer; 126 Adjust Slide Volume With Reaction Buffer; 127 Apply Coverslip; 128 Rinse Slide With Reaction Buffer; 129 Adjust Slide Volume With Reaction Buffer; 130 Apply Coverslip; 131 Rinse Slide With Reaction Buffer; 132 Jet Drain With Reaction Buffer; 133 Apply Two Drops of RED ISH DIG PHE, Apply Coverslip, and Incubate for 8 Minutes; 134 Apply One Drop of RED ISH DIG NAP, and Incubate for 4 Minutes; 135 Apply One Drop of RED ISH DIG FR, and Incubate for [0 Hr 24 Min]; 136 [Red Chromogen]; 137 Apply One Drop of RED ISH DIG FR, and Incubate for 4 Minutes; 138 Rinse Slide With Reaction Buffer; 139 Adjust Slide Volume With Reaction Buffer; 140 Apply Coverslip; 141 Rinse Slide With Reaction Buffer; 142 Adjust Slide Volume With Reaction Buffer; 143 Apply Coverslip; 144 Rinse Slide With Reaction Buffer; 145 Jet Drain With Reaction Buffer;

Referring again to FIG. 12A, rectangles 1205 and 1206 include immobilized oligonucleotides specific to chromosome 7 centromere. The chromosome 7 centromere is that region of DNA found near the middle of chromosome 7 where the sister chromatids come closest in contact. The sequence used for chromosome 7 centromere is 680 base pairs and has a sequence according to that shown in Table 2 (SEQ. ID. NO. 1). Exemplary tiles of chromosome 7 centromere are shown as in Table 2 as Chr 7 Tiles 1-10 (SEQ. ID. NOS. 2-11). Accordingly, a first dot of rectangle 1205 would include a plurality of 50-mers having a sequence according to SEQ. ID. NO. 2. Another dot of rectangle 1205 would include a plurality of 50-mers having a sequence according to SEQ. ID. NO. 3. To the extent that there are more dots in rectangle 1205 than there are distinct tiles, the tiles are repeated.

In this case, the device was designed to detect the presence of a human chromosome 7 centromere probe in a solution. The chromosome 7 centromere probe was manufactured using a region of the chromosome 7 alpha-satellite sequence cloned into a bacterial vector containing antibiotic resistance. The purified plasmid was manufactured to include a DIG hapten according to known methods. As a consequence of the method of manufacture, hapten-labeled oligonucleotide fragments of the plasmid are generated. Some of these fragments are complementary to a portion of the chromosome 7 centromere. Other portions are complementary to other portions of the plasmid vector. In particular, representative tiles of the plasmid sequence are shown in Table 2 (Vector Tiles 1-10; SEQ. ID. NOS. 12-21). These sequences are not unique to the target of this analysis (chromosome 7 centromere) and would instead be indicative of any oligonucleotide manufactured using this particular bacterial vector. Because the vector sequences will be present in a variety of different samples, they can be used as a positive control. Accordingly, the Vector Tiles 1-10 were printed in control regions of the device. In particular, the control sequences were immobilized in the heavy vertical 1207. Because rectangles 1205 and 1206 are colored red, array 1200 memorializes that chromosome 7 centromere probe has hybridized to the immobilized oligonucleotides having a sequence matching chromosome 7 centromere. In this example, the fact that heavy vertical 1207 is evident as black, the device can be interpreted as having plasmid DNA hybridized thereon. If rectangles 1205 and 1206 had not stained, but heavy verticals 1207 had stained, it could be concluded that the chromosome 7 centromere did not hybridize to the device and that the vector sequences had. If neither rectangle 1205 nor heavy vertical 1207 stained, there may be a problem with the staining procedure, the test solution, or the instrument.

TABLE 2
SEQ. ID. NOS. 1-21 provided with name and sequence.

| SEQ. | | |
|---|---|---|
| ID | | |
| NO. | NAME | SEQUENCE |
| 1 | chromosome 7 | AATTCTAAGTAACTTCTTTGTGCTGTGTGTATTCAACTCACAGAGTGGAACGT |
| | centromere | CCCTTTAGACAGAGCAGATTTGAAACACTCTTTTTGTGGAATTTGCAAGTGGA |
| | | GATTTCAAGCGATTTGATGCCAACAGTAGAAAAGGAAATATCTTCAAATAAA |
| | | AACTAGACAGAATCATTCTCAGAAACTACTTTGTGATGTGTGCCTTCAACTCA |
| | | CAGAGTTTAACCTTTCTTTTCTTAGAGCAGTTTAGAAACACTCTGCTTGTTATG |
| | | TCTGCAAGTGGATAACTGGACCTCTTTGAGGCCTTCGTTGCAAACGGGGTTTCT |
| | | TCCTTTCATGCTAGACAGAAGAGTTCTCAGTAACTTTTTTGTGTTGTGTGTATT |
| | | CAACTCACAGAGTTGAACCTTGCTTTAGAGAGAGCAGATTTGAAACACTCTTG |
| | | CTGTGGCATTTTCAGGTGGAGATTTCAAGCGATTTGAGGACAATTGCAGAAAA |
| | | GGAAATATCTTCGTATAACAACCAGACAGAATCATTCTCAGAAAGTGCTTTG |
| | | TGATGTGTGCGTTCAACTCACAAAGTTTAACCTTTCTTTTCATAGAGGAGTTTG |
| | | AAAACACACTGTTTGTAAAGTCTGCAAGTGGATATATGGACCTGTTTGAGGCC |
| | | TTCGTTGGAAACGGGATTTCTTCATTGAATGCTAGACGGAAG |
| 2 | Chr 7-Tile 1 | AATTCTAAGTAACTTCTTTGTGCTGTGTGTATTCAACTCACAGAGTGGAA |
| 3 | Chr 7-Tile 2 | AACTTCTTTGTGCTGTGTGTATTCAACTCACAGAGTGGAACGTCCCTTTA |
| 4 | Chr 7-Tile 3 | TGCTGTGTGTATTCAACTCACAGAGTGGAACGTCCCTTTAGACAGAGCAG |
| 5 | Chr 7-Tile 4 | ATTCAACTCACAGAGTGGAACGTCCCTTTAGACAGAGCAGATTTGAAACA |
| 6 | Chr 7-Tile 5 | CAGAGTGGAACGTCCCTTTAGACAGAGCAGATTTGAAACACTCTTTTTGT |
| 7 | Chr 7-Tile 6 | CGTCCCTTTAGACAGAGCAGATTTGAAACACTCTTTTTGTGGAATTTGCA |
| 8 | Chr 7-Tile 7 | GACAGAGCAGATTTGAAACACTCTTTTTGTGGAATTTGCAAGTGGAGATT |
| 9 | Chr 7-Tile 8 | CTCTTTTTGTGGAATTTGCAAGTGGAGATTTCAAGCGATTTGATGCCAAC |
| 10 | Chr 7-Tile 9 | GGAATTTGCAAGTGGAGATTTCAAGCGATTTGATGCCAACAGTAGAAAAG |
| 11 | Chr 7-Tile 10 | AGTGGAGATTTCAAGCGATTTGATGCCAACAGTAGAAAAGGAAATATCTT |
| 12 | Vector-Tile 1 | TCGCGCGTTTCGGTGATGACGGTGAAAACCTCTGACACATGCAGCTCCCG |
| 13 | Vector-Tile 2 | CGGTGATGACGGTGAAAACCTCTGACACATGCAGCTCCCGGAGACGGTCA |
| 14 | Vector-Tile 3 | GGTGAAAACCTCTGACACATGCAGCTCCCGGAGACGGTCACAGCTTGTCT |
| 15 | Vector-Tile 4 | TCTGACACATGCAGCTCCCGGAGACGGTCACAGCTTGTCTGTAAGCGGAT |
| 16 | Vector-Tile 5 | GCAGCTCCCGGAGACGGTCACAGCTTGTCTGTAAGCGGATGCCGGGAGCA |
| 17 | Vector-Tile 6 | GAGACGGTCACAGCTTGTCTGTAAGCGGATGCCGGGAGCAGACAAGCCCG |
| 18 | Vector-Tile 7 | TCAGCGGGTGTTGGCGGGTGTCGGGGCTGGCTTAACTATGCGGCATCAGA |
| 19 | Vector-Tile 8 | TTGGCGGGTGTCGGGGCTGGCTTAACTATGCGGCATCAGAGCAGATTGTA |
| 20 | Vector-Tile 9 | TCGGGGCTGGCTTAACTATGCGGCATCAGAGCAGATTGTACTGAGAGTGC |
| 21 | Vector-Tile 10 | CTTAACTATGCGGCATCAGAGCAGATTGTACTGAGAGTGCACCATATGCG |

In addition to the chromosome 7 centromere probe, device 1200 of FIG. 12A also includes 20 other stained rectangular features. While not disclosed in detail, these features included immobilized oligonucleotides specific to a multiplicity of other probes present in the tested solution. Likewise, each other array location (each location of both 9 row by 12 column array) also included immobilized oligonucleotides specific to other probes not present in the tested solution. The absence of staining in these array locations provides an indication that those probes were not present in the tested sample. According to illustrative embodiments, the sequences of these immobilized oligonucleotides are selected so that they include the most likely contaminant probes. In particular, if device 1200 was being used as a quality control measure for a mixture of probes including the chromosome 7 centromere probe, other probe sequences that were not to be incorporated into the probe mixture, but present within the facility, may be included on the device to test for unintended mixtures.

Referring again to FIG. 12A, shown is another example of the different characters that may be used on devices according to the present invention. In particular, FIG. 12 A points out a series of characters 1208 patterned on device 1200. FIG. 12B is a magnified top view of device 1200 showing series of characters 1208 in greater detail. FIG. 12 B shows the phrase “WE INNOVATE HEALTHCARE” in a sans serif type-face written in a first size 1210, a second size 1209, and a third size 1211. The smallest type-face (first size 1210) is an approximately 0.2 point font. The second smallest type-face (second size 1209) is an approximately 0.4 point font. The largest type-face (third size 1211) is an approximately 5 point font. Without magnification, visual identification of only the largest type face (third size 1211) is reasonable. However, the resolution of the patterns is so high (approximately 1950 dpi), the 0.2 point font can be read under magnification.

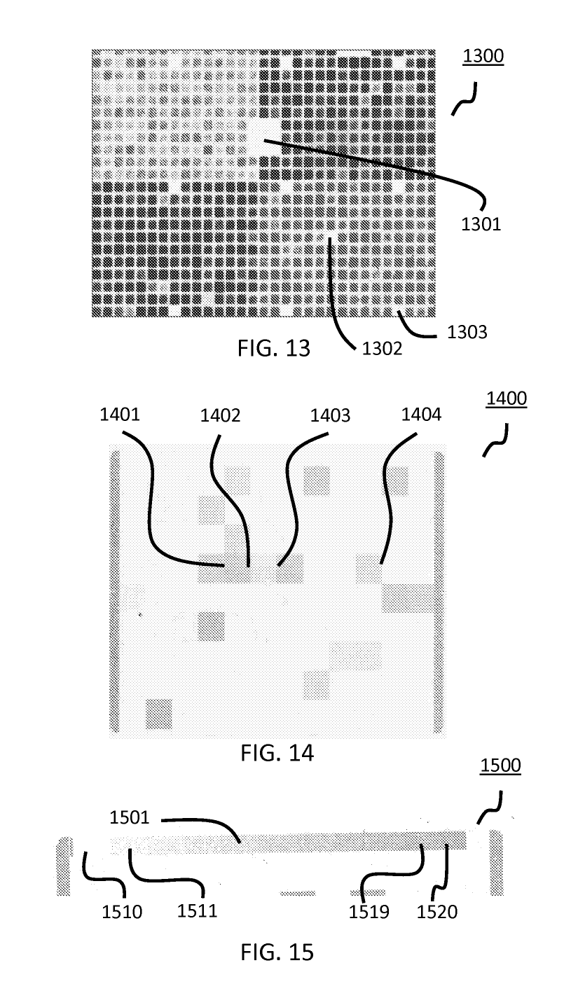

FIG. 13 shows a magnified view of a portion 1300 of an exemplary device showing an array of microscopic features (i.e. dots) that were used to construct the larger characters. The dots are arrayed and are approximately 13 μm by 13 μm in size so that they have an area of approximately 169 μm². Referring to the rectangular characters of FIG. 12A (e.g. rectangles 1205 and 1206), there are approximately 115 dots per the height of a rectangle (1.5 mm). Accordingly, the rectangles of FIG. 12A are patterned on the substrate surface using a typeface with an approximately 4 point font. The resolution in which the pattern was printed is approximately 1950 dpi. Regions 1301, 1302, and 1302 are not stained despite being parts of larger stained characters. An aspect of some devices, such as the exemplary device of which portion 1300 is shown in FIG. 13, is that some dots are reserved by the microarray manufacturers for quality control and positioning functions within their manufacturing process. As such, these regions do not include immobilized oligonucleotides according to the same pattern proscribed for the overall pattern. One aspect of the present disclosure is that the readability of the overall character is not significantly diminished by one or more non-stained dot within the character. Essentially, the dispersion of relatively few non-stained dots (e.g. for quality control) do not prevent the accurate assessment of the characters.

### Example 2

### Reader Interpretation and Reproducibility

In one embodiment, a device according to the present disclosure may be primarily designed to have a binary outcome, in which, if the test and expected pattern match exactly the hypothesized result is confirmed. If the test and expected pattern does not match, for example extra or missing patterns, then the hypothesized result is not confirmed. For testing readability, additional information was gathered on reader dynamic range. For scoring the device, each reader was instructed to match the test outcome pattern with the expected outcome pattern, to determine if the test outcome and expected outcome matched, as well as to determine if there were missing patterns or additional patterns. If there were additional or missing patterns, each reader was given a table, enabling them to identify the missing or additional probe components from the test pattern outcome. They were also asked if they could detect the control features and to count how many black and red characters they observed. The result of this study is shown in Table 3.

aFAT Expected Conditions: ReaderResults

| | | Array | | | | |
|---|---|---|---|---|---|---|
| | | Control | | | Additional | |
| | | Features | # Black | # Red | Signals | Signals |
| | | Present | Signals | Signals | Present | Missing |
| Pattern 1 | Expected | Yes | 20 + 1 | 22 + 1 | No | no |
| | Reader 1 | yes | 20 | 23 | no | no |
| | Reader 2 | yes | 21 | 23 | No | no |
| | Reader 3 | yes | 20 | 22 | no | no |
| Pattern 2 | Expected | Yes | 20 + 1 | 30 + 1 | No | no |
| | Reader 1 | yes | 22 | 28 | No | no |
| | Reader 2 | yes | 20 + 1 | 30 + 1 | no | no |
| | Reader 3 | yes | 20 | 28 | no | no |
| Pattern 3 | Expected | Yes | 20 + 1 | 0 | No | no |
| | Reader 1 | yes | 20 | 0 | No | no |
| | Reader 2* | yes | 20 + 1 | 0 | No | no |
| | | Yes | 21 | 0 | No | no |
| | Reader 3 | yes | 20 | 0 | no | no |
| Pattern 4 | Expected | Yes | 16 + 1 | 0 | No | yes |
| | Reader 1 | yes | 16 | 0 | No | yes |
| | Reader 2 | yes | 16 + 1 | 0 | no | yes |
| | Reader 3 | yes | 16 | 0 | No | yes |
| Pattern 5 | Expected | Yes | 20 + 1 | 2 + 1 | No | no |
| | Reader 1 | yes | 20 | 2 | No | no |
| | Reader 2 | yes | 21 | 3 | No | no |
| | Reader 3 | yes | 20 | 2 | No | no |
| Pattern 6 | Expected | Yes | 20 + 1 | 2 + 1 | No | no |
| | Reader 1 | yes | 20 | 3 | No | no |
| | Reader 2 | yes | 20 + 1 | 2 + 1 | no | no |
| | Reader 3* | yes | 20 | 2 | No | no |
| | | Yes | 20 | 2 | No | no |
| Pattern 7 | Expected | Yes | 20 + 1 | 2 + 1 | No | no |
| | Reader 1* | yes | 21 | 3 | No | no |
| | | Yes | 20 | 2 | No | no |
| | Reader 2 | yes | 21 | 3 | no | no |
| | Reader 3 | yes | 20 | 2 | No | no |
| Pattern 8 | Expected | Yes | 20 + 1 | 2 + 1 | No | no |
| | Reader 1 | yes | 20 | 2 | No | no |
| | Reader 2 | yes | 21 | 2 | no | no |
| | Reader 3 | yes | 20 | 2 | No | no |

A total of 14 devices were stained. Four of which were reread for reader reproducibly, for a total of 18 array slides read by each qualified reader. All devices stained appropriately as expected with no missing control features or staining anomalies. All readers were able to correctly identify a correct expected outcome, as well as if patterns were missing or additional. The mark (*) indicates that the pattern was selected for the reader to read twice.

### Example 3

### Dynamic Range and Gray Scales

Devices were tested for their ability to quantitatively determine the concentration of labeled oligonucleotides in solution. Referring now to FIG. 14, shown is a magnified top view of a device 1400 according to one embodiment of the present invention. Device 1400 shows various concentrations of oligonucleotides in test solution resulted in patterns having colorations with a range of intensities. In particular, the referenced rectangles were examples of oligonucleotide detection and identification at levels according to Table 4.

TABLE 4
Quantitative oligonucleotide patterns.

| Oligonucleotide | |
|---|---|
| microgram | |
| dispensed | Character |
| 2 | heavy bar 1401 |
| 0.007 | rectangle 1402 |
| 0.005 | rectangle 1403 |
| 0.004 | rectangle 1404 |
| 0.003 | rectangle 1405 |

It was determined that procedures developed to detect 2 μg of haptenated oligonucleotide dispensed on the device were capable of detecting 0.2% of that concentration without modification of the procedure. This equates to a dynamic range of approximately 3 orders of magnitude. The procedures identified herein, as described for EXAMPLE 1, were used to test the probe solutions to determine the dynamic range varying only the probe concentration. In particular, a mixture of probes were simultaneously analyzed on a device. The various probes were added at different concentrations to assess the intensity of the resulting staining. While limits of detection are traditionally related to the noise of the measurement, the present calculations deviate from that approach. Since these exemplary devices were designed to be read visually, the limit of detection was based on qualified readers visually detecting staining. Characters having staining intensities that were decipherable visually from the background were assessed as readable.

Referring now to FIG. 15, shown is a magnified top view of a device 1500 according to the present invention showing a gray scale 1501 included in the device. Gray scale 1501 was constructed on the device with 10 different gray scale levels. At one end, level 1510 includes 100% dots having immobilized oligonucleotides specific to a negative control. At the other end, level 1520 includes 100% dots having immobilized oligonucleotides specific to a positive control. Between, these extremes lies a percentage of dots having immobilized oligonucleotides specific to the positive and negative controls steps in increments of 10% so that block 1511 includes 10% of dots having immobilized oligonucleotides specific to a positive control and 90% of the dots include immobilized oligonucleotides specific to a negative control. Similarly, block 1519 includes 10% of dots having immobilized oligonucleotides specific to a negative control and 90% of the dots include immobilized oligonucleotides specific to a positive control. Examined from left to right, gray scale 1501 appears to gradually get darker. As described herein, the color was developed using chromogenic detection which causes staining to occur in those dots which a binding event has occurred.

The gray scale may be used as a quality control measure to reject all characters which have lighter staining than a specific section of the gray scale. For example, those characters that exhibit slight staining may be rejected as being observed or present if the amount of staining is below 10% (e.g. block 1511) on the gray scale. This approach may be implemented when a particular immobilized binding molecule exhibits non-specific binding. While the rectangles having various staining levels and the gray scale appear similar in terms of range of intensity, the manner in which these two features obtain gray staining are distinct. In particular, when a character that includes dots that are 100% directed to the target sequence, the signal should be as dark as gray scale block 1520. However, if there are insufficient amounts of the target oligonucleotide in the test solution, it is possible that only 10% (more or less) of those immobilized oligonucleotides have hybridized to target oligonucleotides. Since only 10% of those immobilized oligonucleotides have bound to a target, the development of color would be expected to be 10% of the maximum. Gray scale 1501 mimics the hybridization of only a portion of the immobilized oligonucleotide by distributing positive control dots which are expected to bind target oligonucleotides at a maximum level with negative control dots which are expected to bind to essentially nothing. The resulting pattern appears gray upon visual inspection without magnification. However, under magnification, the pattern of stained dots distributed amongst un-stained areas would become apparent.

1. A device comprising
a substrate with at least one substrate surface and
a plurality of immobilized detection molecules bound to the substrate surface,
wherein the plurality of immobilized detection molecules are patterned on the substrate surface to form at least one optically decipherable pattern.

2. The device of claim 1, wherein the detection molecules are oligonucleotides or peptides.

3. The device of claim 1, wherein the optically decipherable pattern includes a shape pattern and/or a positional pattern.

4. The device of claim 1, wherein the at least one optically decipherable pattern is a glyph rendered from a character selected from the Universal Character Set, defined by the International Standard ISO/IEC 10646.

5. The device of claim 3, wherein the glyph is associated with a typeface, the typeface having a typographic size of between about 1 and about 216 points or between about 3 and about 96 points, wherein one point is 1/72 of inch.

6. The device of claim 1, wherein the plurality of detection molecules includes a first immobilized detection molecule that is specific to a first target molecule and a second immobilized detection molecule that is specific to a first control molecule.

7. The device of claim 6, wherein the first immobilized detection molecule is patterned on the substrate surface to form a first character and the second immobilized detection molecule is patterned on the substrate surface to form a second character, the characters selected from the Universal Character Set, defined by the International Standard ISO/IEC 10646.

8. The device of claim 7, wherein the plurality of immobilized detection molecules includes a third immobilized detection molecule that is specific to a second target molecule, wherein the third immobilized detection molecule is patterned on the substrate surface to form a third character.

9. The device of claim 7, the first character and the second character cover different areas on the substrate surface.

10. The device of claim 1, wherein the at least one optically decipherable pattern is a glyph rendered from a character selected from the Universal Character Set, defined by the International Standard ISO/IEC 10646 and the glyph comprises a plurality of dots, the plurality of dots having at least one microscopic dimension, wherein a microscopic dimension is less than about 500 μm, less than about 400 μm, less than about 300 μm, less than about 200 μm, between about 1 μm and about 500 μm, or between about 5 and 250 μm.

11. The device of claim 10, wherein the plurality of dots are patterned on the substrate surface at least about 4000 dots per inch, about 3000 dots per inch, about 2000 dots per inch, about 1000 dots per inch, about 600 dots per inch, about 300 dots per inch, about 150 dots per inch about 72 dots per inch, between about 72 and about 4000 dots per inch, between about 150 and about 3000 dots per inch, or between about 300 and about 2000 dots per inch.

12. The device of claim 10, wherein at least about 75%, 90%, 95%, or 99% of the plurality of immobilized detection molecules bound to the substrate surface within each of the plurality of dots have identical molecular structures.

13. The device of claim 1, wherein
the plurality of immobilized detection molecules include a plurality of immobilized oligonucleotides,
the plurality of immobilized oligonucleotides are patterned on the substrate surface to form the at least one optically decipherable pattern rendered as at least one glyph, the at least one glyph comprising a plurality of dots,
at least about 75%, 90%, 95%, or 99% of the plurality of immobilized oligonucleotides bound to the substrate surface within each of the plurality of dots have identical nucleic acid sequences, and
the plurality of immobilized oligonucleotides bound to the substrate surface within each of the at least one glyph are sufficiently complimentary to a first target nucleotide sequence.

14. The device of claim 13, wherein the plurality of immobilized oligonucleotides bound to the substrate surface within the at least one glyph are tiled across the first target nucleotide sequence.

15. The device of claim 13, wherein the plurality of immobilized oligonucleotides are tiled across the first nucleotide sequence in increments of about 1 to 50 base pairs, 2 to 25 base pairs, or 5 to 10 base pairs.

16. The device of claim 13, wherein the first target nucleotide sequence is between about 10 and about 500 base pairs in sequence length and the immobilized oligonucleotides are between about 10 and 100 base pairs in length.

17. The device of claim 1, wherein the at least one optically decipherable pattern is an information code.

18. The device of claim 17, wherein the information code is an optical machine-readable representation of data.

19. The device of claim 17, wherein the information code is a 1-dimensional data code or a 2-dimensional data code.

20. The device of claim 1, wherein the at least one optically decipherable pattern is compatible with automatic identification and data capture.

21. The device of claim 20, wherein the at least one optically decipherable pattern is compatible with optical character recognition, optical mark recognition, or bar code recognition.

22. The device of claim 1, wherein the substrate is glass.

23. The device of claim 1, wherein the substrate is a glass microscope slide.

24. The device of claim 1, wherein the substrate is compatible with use on an automated slide stainer.


SEQUENCE LISTING (21 sequences)

SEQ ID NO: 1 - DNA, 680 bp, Homo sapiens
aattctaagtaacttctttgtgctgtgtgtattcaactcacagagtggaacgtccctttagacagagcagatttgaaacactctttttgtggaatttgcaagtggagatttcaagcgatttgatgccaacagtagaaaaggaaatatcttcaaataaaaactagacagaatcattctcagaaactactttgtgatgtgtgccttcaactcacagagtttaacctttcttttcttagagcagtttagaaacactctgcttgttatgtctgcaagtggataactggacctctttgaggccttcgttgcaaacggggtttcttcctttcatgctagacagaagagttctcagtaacttttttgtgttgtgtgtattcaactcacagagttgaaccttgctttagagagagcagatttgaaacactcttgctgtggcattttcaggtggagatttcaagcgatttgaggacaattgcagaaaaggaaatatcttcgtataacaaccagacagaatcattctcagaaagtgctttgtgatgtgtgcgttcaactcacaaagtttaacctttcttttcatagaggagtttgaaaacacactgtttgtaaagtctgcaagtggatatatggacctgtttgaggccttcgttggaaacgggatttcttcattgaatgctagacggaag

SEQ ID NO: 2 - DNA, 50 bp, Homo sapiens
aattctaagtaacttctttgtgctgtgtgtattcaactcacagagtggaa

SEQ ID NO: 3 - DNA, 50 bp, Homo sapiens
aacttctttgtgctgtgtgtattcaactcacagagtggaacgtcccttta

SEQ ID NO: 4 - DNA, 50 bp, Homo sapiens
tgctgtgtgtattcaactcacagagtggaacgtccctttagacagagcag

SEQ ID NO: 5 - DNA, 50 bp, Homo sapiens
attcaactcacagagtggaacgtccctttagacagagcagatttgaaaca

SEQ ID NO: 6 - DNA, 50 bp, Homo sapiens
cagagtggaacgtccctttagacagagcagatttgaaacactctttttgt

SEQ ID NO: 7 - DNA, 50 bp, Homo sapiens
cgtccctttagacagagcagatttgaaacactctttttgtggaatttgca

SEQ ID NO: 8 - DNA, 50 bp, Homo sapiens
gacagagcagatttgaaacactctttttgtggaatttgcaagtggagatt

SEQ ID NO: 9 - DNA, 50 bp, Homo sapiens
ctctttttgtggaatttgcaagtggagatttcaagcgatttgatgccaac

SEQ ID NO: 10 - DNA, 50 bp, Homo sapiens
ggaatttgcaagtggagatttcaagcgatttgatgccaacagtagaaaag

SEQ ID NO: 11 - DNA, 50 bp, Homo sapiens
agtggagatttcaagcgatttgatgccaacagtagaaaaggaaatatctt

SEQ ID NO: 12 - DNA, 50 bp, Artificial Sequence
  pUCK Vector
tcgcgcgtttcggtgatgacggtgaaaacctctgacacatgcagctcccg

SEQ ID NO: 13 - DNA, 50 bp, Artificial Sequence
  pUCK Vector
cggtgatgacggtgaaaacctctgacacatgcagctcccggagacggtca

SEQ ID NO: 14 - DNA, 50 bp, Artificial Sequence
  pUCK Vector
ggtgaaaacctctgacacatgcagctcccggagacggtcacagcttgtct

SEQ ID NO: 15 - DNA, 50 bp, Artificial Sequence
  pUCK Vector
tctgacacatgcagctcccggagacggtcacagcttgtctgtaagcggat

SEQ ID NO: 16 - DNA, 50 bp, Artificial Sequence
  pUCK Vector
gcagctcccggagacggtcacagcttgtctgtaagcggatgccgggagca

SEQ ID NO: 17 - DNA, 50 bp, Artificial Sequence
  pUCK Vector
gagacggtcacagcttgtctgtaagcggatgccgggagcagacaagcccg

SEQ ID NO: 18 - DNA, 50 bp, Artificial Sequence
  pUCK Vector
tcagcgggtgttggcgggtgtcggggctggcttaactatgcggcatcaga

SEQ ID NO: 19 - DNA, 50 bp, Artificial Sequence
  pUCK Vector
ttggcgggtgtcggggctggcttaactatgcggcatcagagcagattgta

SEQ ID NO: 20 - DNA, 50 bp, Artificial Sequence
  pUCK Vector
tcggggctggcttaactatgcggcatcagagcagattgtactgagagtgc

SEQ ID NO: 21 - DNA, 50 bp, Artificial Sequence
  pUCK Vector
cttaactatgcggcatcagagcagattgtactgagagtgcaccatatgcg